SEQUENCE LISTING (27 sequences)

SEQ ID NO: 1 - protein, 15 aa, Mesocricetus auratus
IGLHDPSHGTLPNGS

SEQ ID NO: 2 - protein, 14 aa, Homo sapiens
IGLHDPTQGTEPNG

SEQ ID NO: 3 - protein, 7 aa, Homo sapiens
WIGLHDP

SEQ ID NO: 4 - protein, 8 aa, Homo sapiens
VWIGLHDP

SEQ ID NO: 5 - protein, 9 aa, Homo sapiens
NVWIGLHDP

SEQ ID NO: 6 - protein, 20 aa, Homo sapiens
CKKSIENAKQDLLQLKNVIS

SEQ ID NO: 7 - protein, 919 aa, Homo sapiens
MTGYTMLRNGGAGNGGQTCMLRWSNRIRLTWLSFTLFVILVFFPLIAHYYLTTLDEADEAGKRIFGPRVGNELCEVKHVLDLCRIRESVSEELLQLEAKRQELNSEIAKLNLKIEACKKSIENAKQDLLQLKNVISQTEHSYKELMAQNQPKLSLPIRLLPEKDDAGLPPPKATRGCRLHNCFDYSRCPLTSGFPVYVYDSDQFVFGSYLDPLVKQAFQATARANVYVTENADIACLYVILVGEMQEPVVLRPAELEKQLYSLPHWRTDGHNHVIINLSRKSDTQNLLYNVSTGRAMVAQSTFYTVQYRPGFDLVVSPLVHAMSEPNFMEIPPQVPVKRKYLFTFQGEKIESLRSSLQEARSFEEEMEGDPPADYDDRIIATLKAVQDSKLDQVLVEFTCKNQPKPSLPTEWALCGEREDRLELLKLSTFALIITPGDPRLVISSGCATRLFEALEVGAVPVVLGEQVQLPYQDMLQWNEAALVVPKPRVTEVHFLLRSLSDSDLLAMRRQGRFLWETYFSTADSIFNTVLAMIRTRIQIPAAPIREEAAAEIPHRSGKAAGTDPNMADNGDLDLGPVETEPPYASPRYLRNFTLTVTDFYRSWNCAPGPFHLFPHTPFDPVLPSEAKFLGSGTGFRPIGGGAGGSGKEFQAALGGNVPREQFTVVMLTYEREEVLMNSLERLNGLPYLNKVVVVWNSPKLPSEDLLWPDIGVPIMVVRTEKNSLNNRFLPWNEIETEAILSIDDDAHLRHDEIMFGFRVWREARDRIVGFPGRYHAWDIPHQSWLYNSNYSCELSMVLTGAAFFHKYYAYLYSYVMPQAIRDMVDEYINCEDIAMNFLVSHITRKPPIKVTSRWTFRCPGCPQALSHDDSHFHERHKCINFFVKVYGYMPLLYTQFRVDSVLFKTRLPHDKTKCFKFI

SEQ ID NO: 8 - protein, 15 aa, Artificial
  synthetic
  ACETYLATION (1)..(1)
  AMIDATION (15)..(15)
IGLHDPSHGTLPNGS

SEQ ID NO: 9 - protein, 15 aa, Artificial
  synthetic
  ACETYLATION (1)..(1)
  PEGYLATION (15)..(15): conjugation with 40-kDa poly (ethylene glycol) (PEG)-maleimide
  AMIDATION (15)..(15)
IGLHDPSHGTLPNGS

SEQ ID NO: 10 - protein, 15 aa, Artificial
  synthetic
  PEGYLATION (1)..(1): conjugation with 40-kDa poly (ethylene glycol) (PEG)-maleimide-3-mercaptopropionic acid
  AMIDATION (15)..(15)
IGLHDPSHGTLPNGS

SEQ ID NO: 11 - protein, 17 aa, Artificial
  synthetic
  MISC_FEATURE: Cyclic amide bond between side chain of Asp on position 1 and Lys on position 17
  ACETYLATION (1)..(1)
  AMIDATION (17)..(17)
DIGLHDPSHGTLPNGSK

SEQ ID NO: 12 - protein, 14 aa, Artificial
  synthetic
  ACETYLATION (1)..(1)
  AMIDATION (14)..(14)
IGLHDPTQGTEPNG

SEQ ID NO: 13 - protein, 14 aa, Artificial
  synthetic
  ACETYLATION (1)..(1)
  PEGYLATION (14)..(14): conjugation with 40-kDa poly (ethylene glycol) (PEG)-maleimide
  AMIDATION (14)..(14)
IGLHDPTQGTEPNG

SEQ ID NO: 14 - protein, 14 aa, Artificial
  synthetic
  PEGYLATION (1)..(1): conjugation with 40-kDa poly (ethylene glycol) (PEG)-maleimide-3-mercaptopropionic acid
  AMIDATION (14)..(14)
IGLHDPTQGTEPNG

SEQ ID NO: 15 - protein, 16 aa, Artificial
  synthetic
  MISC_FEATURE: Cyclic amide bond between side chain of Asp on position 1 and Lys on position 16
  ACETYLATION (1)..(1)
  AMIDATION (16)..(16)
DIGLHDPTQGTEPNGK

SEQ ID NO: 16 - protein, 7 aa, Artificial
  synthetic
  ACETLYATION (1)..(1)
  AMIDATION (7)..(7)
WIGLHDP

SEQ ID NO: 17 - protein, 7 aa, Artificial
  synthetic
  ACETYLATION (1)..(1)
  PEGYLATION (7)..(7): conjugation with 40-kDa poly (ethylene glycol) (PEG)-maleimide
  AMIDATION (7)..(7)
WIGLHDP

SEQ ID NO: 18 - protein, 7 aa, Artificial
  synthetic
  PEGYLATION (1)..(1): conjugation with 40-kDa poly (ethylene glycol) (PEG)-maleimide-3-mercaptopropionic acid
  AMIDATION (7)..(7)
WIGLHDP

SEQ ID NO: 19 - protein, 9 aa, Artificial
  synthetic
  MISC_FEATURE: Cyclic amide bond between side chain of Asp on position 1 and Lys on position 9
  ACETYLATION (1)..(1)
  AMIDATION (9)..(9)
DWIGLHDPK

SEQ ID NO: 20 - protein, 8 aa, Artificial
  synthetic
  ACETLYATION (1)..(1)
  AMIDATION (8)..(8)
VWIGLHDP

SEQ ID NO: 21 - protein, 8 aa, Artificial
  synthetic
  ACETYLATION (1)..(1)
  PEGYLATION (8)..(8): conjugation with 40-kDa poly (ethylene glycol) (PEG)-maleimide
  AMIDATION (8)..(8)
VWIGLHDP

SEQ ID NO: 22 - protein, 8 aa, Artificial
  synthetic
  PEGYLATION (1)..(1): conjugation with 40-kDa poly (ethylene glycol) (PEG)-maleimide-3-mercaptopropionic acid
  AMIDATION (8)..(8)
VWIGLHDP

SEQ ID NO: 23 - protein, 10 aa, Artificial
  synthetic
  MISC_FEATURE: Cyclic amide bond between side chain of Asp on position 1 and Lys on position 10
  ACETYLATION (1)..(1)
  AMIDATION (10)..(10)
DVWIGLHDPK

SEQ ID NO: 24 - protein, 9 aa, Artificial
  synthetic
  ACETYLATION (1)..(1)
  AMIDATION (9)..(9)
NVWIGLHDP

SEQ ID NO: 25 - protein, 9 aa, Artificial
  synthetic
  ACETYLATION (1)..(1)
  PEGYLATION (9)..(9): conjugation with 40-kDa poly (ethylene glycol) (PEG)-maleimide
  AMIDATION (9)..(9)
NVWIGLHDP

SEQ ID NO: 26 - protein, 9 aa, Artificial
  synthetic
  PEGYLATION (1)..(1): conjugation with 40-kDa poly (ethylene glycol) (PEG)-maleimide-3-mercaptopropionic acid
  AMIDATION (9)..(9)
NVWIGLHDP

SEQ ID NO: 27 - protein, 11 aa, Artificial
  synthetic
  MISC_FEATURE: Cyclic amide bond between side chain of Asp on position 1 and Lys on position 11
  ACETYLATION (1)..(1)
  AMIDATION (11)..(11)
DNVWIGLHDPK

(19) United States
(12) Patent Application Publication
(10) Pub. No.: US 2021/0371478 A1
(43) Pub. Date: Dec. 2, 2021

(54) GENERATION OF BRAIN AND SPINAL CORD NEURONS, CARDIAC MYOCYTES, AND HEPATOCYTES USING REG PEPTIDES, PEPTIDOMIMETICS, SMALL MOLECULES AND STIMULATORY ANTIBODIES TO REG RECEPTOR

(71) Applicant: Claresa Levetan, Bryn Mawr, PA (US)

(72) Inventor: Claresa Levetan, Bryn Mawr, PA (US)

(21) Appl. No.: 17/391,883

(22) Filed: Aug. 2, 2021

Related U.S. Application Data
(63) Continuation-in-part of application No. 16/511,718, filed on Jul. 15, 2019.
Application No. 16/511,718 is a continuation of application No. 15/369,685, filed on Dec. 5, 2016, now abandoned.
Application No. 15/369,685 is a continuation-in-part of application No. 13/662,245, filed on Oct. 26, 2012, now Pat. No. 9,511,110.

(51) Int. Cl. C07K 14/47; A61K 35/12; C07K 16/28; A61K 38/08

(52) U.S. Cl. CPC C07K 14/47; A61K 35/12; A61K 38/00; A61K 38/08; C07K 16/2839

(57) ABSTRACT

Reg gene receptors are found throughout the body, including in the neurons of the brain and spinal cord, liver and heart. Reg proteins are expressed during fetal development for organogenesis and then only upregulated in times of organ injury, such as in the setting of stroke, myocardial infarction or spinal cord injury. Upregulation of Reg proteins following organ injury is a protective mechanism against organ failure and has been shown to result in the formation of new neurons, cardiac myocytes, hepatocytes and in other organs expressing the Reg receptor. Described are the compositions of bioactive Reg peptides, as well as optimization of these peptides (to increase plasma half-life), peptidomimetics, stimulatory antibodies and small molecules that interact with the Reg receptor that are capable of initiating formation of new cells after organ injury.

### CROSS-REFERENCE TO RELATED APPLICATIONS

The present application is a Continuation-in-Part of U.S. patent application Ser. No. 16/511,718 filed Jul. 15, 2019, which is a Continuation of U.S. patent application Ser. No. 15/369,685 filed Dec. 5, 2016, which is a Continuation-in-Part of U.S. patent application Ser. No. 13/662,245 filed Oct. 26, 2012, which published as U.S. Patent Application Publication No. 2014/0120097 and is now U.S. Pat. No. 9,511,110, the disclosure of which are all incorporated herein by reference in its entirety.

### REFERENCE TO SEQUENCE LISTING SUBMITTED IN COMPUTER READABLE FORM

The present application contains a Sequence Listing which has been submitted in ASCII format by way of EFS-Web and is hereby incorporated by reference herein in its entirety. The ASCII file was created on Nov. 16, 2016 and named C.LEV-017_seqlist1_ST25, which is 16.4 kb in size and which is identical to the paper copy filed with this application.

### FIELD OF THE INVENTION

The present invention relates to Reg gene receptors and Reg gene protein that bind to the Reg receptors resulting in new neurons of the brain and spinal cord and periphery, cardiac myocytes, hepatocytes and pancreatic ductal cells after organ injury. Reg genes are typically expressed only during fetal development, but are again upregulated in times of organ injury, such as in the setting of stroke, myocardial infarction or spinal cord injury. Upregulation of Reg genes occur immediately following organ injury throughout the body in mammals and humans. Reg receptors are found throughout the body (FIG. 1).

Reg genes were initially ascribed to as being pathologic but have been shown to be a protective mechanism against organ failure. Reg proteins initiate regeneration of new neurons of brain and spinal cord and peripheral neurons, cardiac myocytes and hepatocytes from progenitor cells within organs expressing the Reg receptor as shown in vitro and in vivo in mammalian and human studies. Reg receptors are demonstrated throughout the body (FIG. 1). Reg proteins are highly expressed during fetal organogenesis, but postnatally, the Reg proteins are not detectable unless there is organ injury (Fang et al., Anat Rec. 2010; 293:464-476, Kiji et al., J Vasc Res. 2003; 40:132-139, Orelle et al., J Clin Invest. 1992; 90(6):2284-91, Terazono et al., Am J Physiol Heart Circ Physiol. 2005; 289:H277-H284, Iovanna et al., Am J Physiol. 1991; 261(3 Pt 1):G485-9, Ortiz et al., Gastroenterology. 1998; 114:808-816).

This invention describes compositions of short bioactive Reg peptides and optimized Reg peptides (7-15-amino acids) that bind to the Reg receptor initiating the formation of neurons, myocytes and hepatocytes from progenitor cells within an injured organ expressing the Reg receptor. Optimized Reg peptides refer to the use conventional methods of improving peptide half-life and reducing the therapeutic dosage requirements and include for example, N-terminal acetylation and C-terminal amide and utilized in this invention for the formation of new cells in organs with acute damage, which express Reg receptors. Shorter optimized Reg peptides are also described that have been shown by this inventor to bind to the Reg receptor in man and mammals, both in vitro and in vivo.

While hemopoietic stem cells have not been shown to transdifferentiate into pancreatic islets, cardiac myocytes, neurons or hepatocytes, progenitor cells within organs expressing the Reg receptor have been shown to form new islets within the pancreas, hepatocytes within the liver, peripheral neurons and neurons of the brain and spinal cord and myocytes of the heart, progenitor cells within the brain, spinal cord, liver and heart have been shown to form new neurons, myocytes, hepatocytes and other cells following injury in organs that have Reg receptors (Butler et al., Diabetes. 2007; 56(7):1810-6, Murry et al., Nature. 2004; 8; 428(6983):664-8, Roybon et al., Stem Cells. 2006; 24(6):1594-604). This invention describes usage of Reg peptides and optimized Reg peptides as therapeutics for new cell formation after injury in organs, which express Reg receptors.

The Regenerating (Reg) protein family comprises C-type lectin-like proteins discovered during pancreatitis and pancreatic injury when new pancreatic islets are generated. Reg proteins have numerous names in the literature including pancreatic thread protein (PTP), islet cells regeneration factor, pancreatic stone protein (PSP), and lithostathine. Studies from many different organ injuries provide evidence that Reg proteins signal the proliferation and generation of many cell types, depending on the organ of injury, by binding to the Reg receptor.

This invention describes the usage of shorter and optimized Reg peptide sequences within the mammalian and human Reg gene proteins that were previously patented by this inventor for the formation of new pancreatic islets, and new to the art is usage of Reg peptides and optimized Reg peptides for the generation of new brain, spinal cord and peripheral neurons, hepatocytes and myocytes.

Mammalian Reg proteins have been shown to induce an upregulation of proteins involved in brain, peripheral and spinal cord neuron regeneration. The ability of the 15 amino-acid Reg peptide to improve nerve function and enhance regeneration in streptozotocin-induced diabetic mice was shown two decades ago as coincident to treatment of diabetic mice, yet clinical usage in man or conditions of peripheral nerve aberrations, is new to the art and not described as a potential useful treatment for diabetic neuropathy or other conditions of neuronal loss, nor was the concept that a shorter Reg peptides or optimized Reg peptides considered by, this inventor or others in the art as potentially useful for regeneration of neurons in diabetes, brain or spinal cord neuron, nor has there been prior consideration in the art for usage of Reg peptides, optimized Reg peptides, peptidomimetics, small molecules or stimulating antibodies to generate new cells in injured organs that possess the Reg receptor.

The structures of the human Reg gene proteins are highly conserved with similar sequences within the binding region in both mammals and humans that is the binding region for the Reg receptor. Human Reg1a contains 166-amino acids. Human Reg1b contains 166-amino acids. Human Reg3a contains 174-amino acids. Human Reg4 contains 158-amino acids and a 175-amino acid mammalian Reg protein.

This invention identifies peptide sequences that are within the human Reg1a, human Reg1b, human Reg3a and human Reg4, and other homologous mammalian peptides that are 7-15-amino acids in length and embodiments thereof, that interact with the Reg receptor. This invention specifically demonstrates that homologous peptides that are highly conserved within the human Reg1a, human Reg1b, human Reg3a and Reg4 gene protein and other mammalian Reg peptides that interact with the Reg receptor resulting in the acceleration of the generation of myocytes, hepatocytes and adult brain and spinal cord neurons from progenitor cells within the human brain, liver, spinal cord neurons and the heart and how the data of shorter Reg peptides interacting with the Reg receptor in vitro in human cell lines, in vivo in numerous preclinical and clinical studies using shorter Reg peptides and optimized Reg peptides are translatable to for injury to the brain, spinal cord, heart and other organs that express the Reg receptor.

This inventor has also identified the human Reg receptor, EXTL-3, and in this patent identifies a 20-amino acid binding region within the 919-amino acid Reg receptor/EXTL-3, from which peptidomimetics, small molecules and stimulatory antibodies are generated to the bioactive region of Reg receptor. This inventor has demonstrated how shorter and optimized Reg peptides bind to the Reg receptor resulting in a conformational change to the Reg receptor triggering expression of downstream factors leading to the generation of new cells specifically based on the organ of injury, without the use of embryonic stem cells, but rather the utilizing progenitor cells found within organs, including the brain and spinal cord neurons, liver hepatocytes and cardiac myocytes.

It has been known for decades that acute injury to an organ triggers new cell growth by the upregulation of reparative genes, as a means of protection. With the advent of the Human Genome Project and proteome, this inventor has identified homologous peptide bioactive sequences within the Reg family of gene proteins in man and mammals that bind to the Reg receptor. Bioactive regions within human and mammalian Reg gene proteins, comprising 7-15-amino acid sequences have been patented by this inventor for their capacity for use in initiating the regeneration of pancreatic islets and beta cells for use in diabetes by binding to the human Reg receptor (Levetan, Endocr Pract. 2008; 14(9):1075-1083).

This inventor provides data that during acute injury in different organs including the liver, myocardium and brain and spinal cord neurons, resulting in upregulation of the Reg proteins in organs that express the Reg gene receptor, and new to the art is using Reg peptides and optimized Reg peptides for organs that have shown improved outcomes when Reg proteins are administered. Similar to new islet formation in diabetes, the entire Reg protein is not required for production of new cells within an organ expressing the Reg receptor.

Both in vivo and in vitro in human cell lines, in animal models and human data has shown of the efficacy Reg proteins and shorter Reg peptides in binding to Reg receptor triggering a downstream cascade of factors necessary for generation of new cells (Levetan. J Diabetes. 2010; 2(2):76-84, Levetan Endocr Pract. 2008; 14(9):1075-83). This invention utilizes the shorter Reg peptides and optimized Reg peptides for regeneration of cardiac myocytes, liver hepatocytes, brain and spinal cord neurons peripheral neurons with their efficacy by binding to the Reg receptor initiating the generation of new cells when there is organ damage including brain neurons, spinal cord neurons; and generation of new liver hepatocytes and myocardial cells after myocardial injury.

Therapies described herein may be used with other medications used in the treatment of neurological, cardiac and other disease states for which there is organ damage in which the Reg receptor is expressed. Reg proteins have been shown to be upregulated following organ injury and not expressed in normal liver, heart, pancreas or brain and are found to be protective and regenerative in organ systems expressing the Reg receptor (Fang et al., Anat Rec. 2010; 293:464-476, Kiji et al., J Vasc Res. 2003; 40:132-139, Orelle et al., J Clin Invest. 1992; 90(6):2284-91, Terazono et al., Am J Physiol Heart Circ Physiol. 2005; 289:H277-H284, Iovanna et al., Am J Physiol. 1991; 261(3 Pt 1):G485-9, Ortiz et al., Gastroenterology. 1998; 114:808-816).

Previously, this inventor has shown that EXTL-3 is the Reg receptor in man and that there are peptide sequences within the human Reg1a, Reg1b, Reg3a and Reg4 gene proteins that are homologous 14 and 15-amino acid (Levetan, Endocr Pract. 2008; 14(9):1075-1083) (FIGS. 2a and b)

This invention demonstrates that 7-15-amino acid sequences of Reg proteins interact with the Reg receptor, EXTL-3, which is a cell surface receptor, resulting in downstream activation to the cell nucleus with upregulation of transcription factors resulting in the formation of new functional tissue in organs other than pancreas (Levetan Endocr Pract. 2008; 14(9):1075-1083 and U.S. Pat. Nos. 8,911,776; 8,816,047; 7,989,415; 7,393,919; 9,133,440; 8,829,158; 8,785,400; 8,383,578; 8,211,430; 7,989,412; 7,714,103; 7,393,919; 8,816,047; and 9,321,812).

This invention utilizes 7-15-amino peptides and 7-15-amino acid optimized Reg peptides for regeneration of cardiac myocytes, brain, spinal cord and peripheral neurons, liver hepatocytes and cardiac myocytes and other organs in which there is acute and chronic injury for which Reg proteins bind to the Reg receptor triggering downstream production of the cells. Reg proteins are upregulated during acute injury and induce the formation of new cells from progenitor cells within the organ of injury.

This inventor and others have shown that Reg gene proteins that are typically only expressed during embryonic development, when Reg is critical in the formation of developing organs. After fetal development, the Reg proteins are downregulated, but have been found to be highly expressed in times of acute injury of the brain, spinal cord, liver and heart (Levetan. J Diabetes. 2010; 2(2):76-84, Levetan Endocr Pract. 2008; 14(9):1075-83).

This invention identifies a 20-amino acid binding region on the Reg receptor also known as EXTL-3, which has 100% homology in man and among 17 other mammals. Shorter Reg peptides and optimized Reg peptides have been shown by this inventor to interact with the Reg receptor and new to the art, may be used in treatment of neurological, spinal cord and myocardial damage, and other organs that express the Reg receptor following injury.

### BACKGROUND OF THE INVENTION

Mammalian Reg proteins have been shown to induce an upregulation of proteins involved in brain, peripheral and spinal cord neuron regeneration. The ability of the 15 amino-acid Reg peptide to improve nerve function and enhance regeneration in streptozotocin-induced diabetic mice was shown two decades ago as coincident to treatment of diabetic mice, yet clinical usage in man or conditions of peripheral nerve aberrations, is new to the art and not described as a potential useful treatment for diabetic neuropathy or other conditions of neuronal loss, nor was the concept that a shorter Reg peptides or optimized Reg peptides considered by this inventor or others in the art as potentially useful for regeneration of neurons in diabetes, brain or spinal cord neurons, nor has there been prior consideration in the art for usage of Reg peptides, optimized Reg peptides, peptidomimetics, small molecules or stimulating antibodies to generate new cells in injured organs that possess the Reg receptor.

New to the art, this invention provides data to support that usage of shorter Reg peptides acting through the interaction of Reg peptides and optimized Reg peptides through their interaction with the human Reg receptor and may be used in the treatment of injured organs through the generation of new cells. Although, initially thought to be deleterious because Reg was found at the site of acute tissue damage, it is now known that Reg and shorter Reg peptides and optimized Reg peptides are helpful in the treatment of disease states including diabetes by upregulation of transcription factors including that PDX-1, NGN3, NeurodD1, Pax4, MafA, Nkx2.2, Nkx6.1, B4n4, MafB, Pax6, Nkx6.1 and Sox9 (Levetan. J Diabetes. 2010. 2(2):76-84, Levetan Endocr Pract. 2008; 14(9):1075-83, Kapur Islets. 2012; 4(1):40-8., Li Peptides. 2009; 30(12):2242-9, Assouline-Thomas Differentiation. 2015; 90(4-5):77-90). Data demonstrates the role of Reg in many other organs following injury initiating regeneration of new neurons, hepatocytes and myocytes when there is damage to the heart, liver, brain, spinal cord and peripheral neurons.

It has been shown in the brain, spinal cord, liver, pancreas and heart that Reg proteins are expressed in the central nervous system primarily during organ development in utero and necessary, for example in fetal brain development, while Reg proteins are absent in the adult brain, heart pancreas and liver, but following acute injury or stress Reg proteins are again expressed (Fang et al., Anat Rec. 2010; 293:464-476, Kiji et al., J Vasc Res. 2003; 40:132-139, Orelle et al., J Clin Invest. 1992; 90(6):2284-91, Terazono et al., Am J Physiol Heart Circ Physiol. 2005; 289:H277-H284, Iovanna et al., Am J Physiol. 1991; 261(3 Pt 1):G485-9, Ortiz et al., Gastroenterology. 1998; 114:808-816).

While the increased presence of Reg protein in many conditions including new onset diabetes and Alzheimer's disease (AD), acute myocardial infarction and models of brain and spinal cord injury and in diabetic peripheral neuropathy was initially thought to be part of the destructive process, Reg proteins have emerged as anti-inflammatory, antiapoptotic and mitogenic within multiple organs and are specifically found to be expressed in organ of injury that express the Reg receptor.

Reg protein has been shown to be overexpressed during the very early stages of Alzheimer disease, with deposits detected in the brain of patients with Alzheimer disease, but initially the function of Reg was unclear, but was shown to significant increase the number of cells with longer neurites in neuronal cell lines in rat primary hippocampal neurons and specifically shown to mediate its effects through the Reg receptor, EXTL-3 (Acquatella-Tran Van Ba et al., J Biol Chem. 2012; 287:4726-4739).

Reg initiates neuronal regeneration with up to a 30-fold increased expression of Reg protein in brain tissue of humans with Alzheimer's disease compared to healthy controls in the very early course of disease (de la Monte et al., J. Clin. Investig. 2010; 86, 1004-1013, Duplan et al., Neurobiol Aging 2001; 22(1):79-88) while typically the Reg receptor is expressed in the developing mouse cerebral (Osman et al., Brain Res. Dev. Brain Res. 2004; 151, 111-117). In the mature brain, Reg was associated with neuronal sprouting and regeneration (Ozturk et al., Proc. NatL. Acad. Sci. USA. 1989; 86:419-423, Hashimoto et al., Neurol. Sci. 2003, 138, 26-35 16).

A 15-amino acid Reg peptide that has been used in man and shown to generate new insulin-producing beta cells has also been shown to enhance nerve growth from sensory ganglia alone or in combination with insulin in diabetic mice with peripheral neuropathy with mice rendered diabetic via streptozotocin. The 15-amino acid Reg peptide has demonstrated following a 2-wk treatment and dorsal root ganglia removed from the groups with and without treatment with the demonstrated enhanced nerve outgrowth in the group treated with the shorter Reg peptide (Tam et al., FASEB J. 2004; 18(14):1767-9, Tam et al., Biochem Biophys Res Commun. 2002; 291(3):649-54). The ability of the Reg peptide to generate new neurons correlated with an increase in [(3)H]thymidine incorporation (P<0.0001) and mitochondrial activity (P<0.001) with the findings that the Reg peptide may promote Schwann cell proliferation in the dorsal root ganglia promoting neurite outgrowth.

Western blotting experiments revealed attenuation of neurofilament hyperphosphorylation, up-regulation of beta-tubulin and actin, and increased phosphorylation of the transcription factor STAT3 in dorsal root ganglia of treated diabetic mice with findings that the 15 amino-acid Reg peptide activates signaling pathways implicated in nerve regeneration in the sensory ganglia of the peripheral nervous system. These studies have implications for not only diabetic peripheral neuropathy, but other forms of neuron growth.

The expression of Reg in the neonatal brain was studied in a pediatric model of traumatic brain injury in newborn pups and found that Reg HIP/PAP has strong neuroprotective/neuroregenerative potential following excitotoxic injury to the developing brain, and could represent an interesting therapeutic strategy in perinatal brain injury (Halipur et al., Ann Clin Transl Neurol. 2014; 1(10):739-54).

Reg has been shown to be a motoneuron trophic factor signaling neuron survival in rats (Nishimune et al., Nat Cell Biol. 2000; 2(12):906-14) with expression of Reg after peripheral nerve injury in rats is sensory neuron protective (Averill et al., J. Neurosci. 2002; 22, 7493-7501).

The neuroprotective effectives of Reg in a rodent model of spinal cord transection injury at the ninth thoracic level found that Reg treatment significantly reduced neuronal death in the spinal cord (Fang et al., Anat Rec. 2010; 293(3):464-76, Fang et al., Anat Rec. 2011; 294(1):24-45). There was also an attenuation of inflammation at the injury site and an increase in white matter sparing and retained myelination. Retrograde tracing revealed that Reg protected axons of long descending pathways at 6 weeks post-spinal cord injury, and the number of FluoroGold-labeled neurons in spinal and supraspinal regions was also significantly increased. Immunofluorescent staining confirmed that the spared white matter contained neurofilament-positive axons. Moreover, behavioral improvements as measured by Basso Beattie Bresnahan locomotor rating scores and grid-walk analysis found that Reg treatment might promote functional recovery by increasing axonal growth, inhibiting neuronal apoptosis, and attenuating spinal cord secondary injury after spinal cord injury.

In vitro studies demonstrating the role for Reg in neuron protection and generation include findings that neurite outgrowth activity was significantly suppressed by preincubation with antibodies against Reg and that Reg protein served as a scaffold for neurites and promoted neurite outgrowth (Konishi et al., J Biol Chem. 2013; 288(15):10205-13).

A Reg suppression study by an adenovirus-mediated small interference Reg RNA expression in isolated sciatic nerves, which successfully suppressed Reg found that in crushed sciatic nerves of rats, the suppression of Reg resulted in diminished nerve regeneration with findings that Reg stimulated nerve regeneration (Namikawa et al, J Neurosci. 2006; 26(28):7460-7).

The regenerating gene (Reg) has been documented to play an important role in various regenerating tissues including the heart. Reg protein was found to expressed in the cytoplasm of cardiomyocytes in human hearts of autopsied patients who died from myocardial infarction and later shown that Reg protein could be activated in response to heart stress and rats and Reg was quantified after coronary artery ligation (Kiji et al., J Vasc. Res. 2003; 40:132-139, Kiji et al., Am J Physiol Heart Circ Physiol. 2005; 289:H277-H284).

Reg protein mRNA activation, peaked at 12 hours after coronary ligation resulting in myocardial ischemia and was seven-fold higher compared with hearts with only pressure overload due to aortic constriction. Reg receptor mRNA was expressed intensely in damaged ventricles of rats with. Western blot analysis and circulating levels of Reg protein after myocardial infarction were significantly higher after coronary artery ligation (Kiji et al. J Vasc Res. 2003; 40:132-139, Kiji et al., Am J Physiol Heart Circ Physiol. 2005; 289:H277-H284).

Further study demonstrated that in surviving cardiomyocytes, the Reg protein-dependent cascade recruits reparative macrophages and promotes neutrophil clearance, which followed a combination of in vivo and in vitro studies found that cardiomyocytes promote repair by releasing Reg protein (Frangogiannis N G, Cell Metab. 2015; 21(6); 6797-798, Lorchner et al., Nat Med, 2015; 21(353-362).

Reg has been shown to accelerates liver regeneration in two studies using Reg transgenic mouse models. A Reg transgenic mouse model demonstrated that 80% of the Reg mice vs. 25% of the control mice were protected against lethal acetaminophen-induced fulminate hepatitis with a single injection of recombinant Reg. Reg was shown to prevent mitochondrial damage by acetaminophen overdose with the conclusions that Reg is a promising candidate for the prevention and treatment of liver failure. (Lieu et al., Hepatology 0.2005; 42:618-626). Another transgenic mouse model after partial hepatectomy also found Reg to be a promising candidate to treat liver failure stimulated liver regeneration after partial hepatectomy (Simon et al., FASEB J. 2003; 17:1441-1450).

This invention specifically identifies Reg peptide sequences previously patented by this inventor, but now for the in vivo development of new cardiac myocytes, brain neurons, nephrons and hepatocytes from the liver or by Reg initiated ex-vivo generation of neurons, cardiac myocytes and hepatocyte generated from progenitor cells from an organ that expresses the Reg receptor or tissue from an organ that expresses Reg and to be delivered directly or indirectly to a patient with injury to an organ expressing Reg.

This inventor demonstrates the use of Reg peptides, small molecules, and stimulating antibodies designed to stimulate the 20-amino acid Reg binding site of the 919-amino acid Reg receptor to be used as therapy in conditions including, but not limited to acute injury to organs, including the brain, central and peripheral nerves, spinal cord injury, stroke and other debilitating neurological conditions in which new neurons and neuron outgrowth can be beneficial, acute myocardial ischemia, myocarditis, cardiomyopathy, diminished cardiac ejection fraction, congestive heart failure, injury of the liver from tumors, toxins, cirrhosis, hepatitis or other conditions resulting in hepatic failure or insufficiency, and acute and chronic organ damage in organs that express the Reg receptor.

This inventor has also raised antibodies to this 20-amino acid binding region of the Reg/EXTL-3 receptor, for which stimulating antibodies and peptidomimetics can be generated to form new cardiac myocytes, new neurons and elongation of existing neurons and hepatocytes resulting from peptides described herein. This invention includes peptidomimetics and stimulatory antibodies to the Reg receptor, EXTL-3 for usage to generate new neurons, nephrons, hepatocytes and cardiac myocytes.

Though there is a great deal of data on the role of Reg proteins in regenerating new cells and data in diabetes and diabetic peripheral neuropathy that shorter Reg peptides result in new cell formation, this has not previously been described in the art with this inventor finding that shorter Reg peptides and optimized Reg peptides interact the Reg/EXTL-3 receptor and may be used in injured brain, cardiac and liver tissue, which include motor neurons, spinal cord, peripheral nerves and myocardium.

Reg gene proteins are typically only expressed during embryonic development, when they are critical in the formation of pancreatic islets, neurons, myocardial cells and other developing organs being generated for the first time. Expression data demonstrates that after fetal development, many organs express the Reg receptor, but Reg proteins are downregulated, but have been found to be highly expressed in times of acute injury Levetan Endocr Pract. 2008; 14(9):1075-83, Levetan C., 2010, J Diabetes; 2(2):76-84) including in the human heart, when the Reg gene protein was shown to be upregulated in response to acute myocardial ischemia (Kiji, Am J Physiol Heart Circ Physiol. 2005; 289(1):H277-84).

Reg gene proteins have been shown to be essential for the survival and neurite outgrowth of spinal cord neurons, with upregulation of the Reg receptor and Reg playing a key a role in protection of spinal cord neurons against injury (Parikh, Biomol Concepts, 2012; 3(1):57-70., Acquatella-Tran Van Ba, J Biol Chem. 2012:10; 287(7):4726-39., Fang. Anat Rec. 2010; 293(3):464-76, Fang, Anat Rec 2011; 294(1):24-45). Fang has specifically shown the role of Reg in protecting neurons after spinal cord transection to promote functional recovery by increasing axonal growth, inhibiting neuronal apoptosis, and attenuating spinal cord secondary injury after spinal cord injury. (Parikh, Biomol Concepts, 2012; 3(1):57-70., Van Ba et al, J Biol Chem. 2012:10; 287(7):4726-39., Fang et al., Anat Rec. 2010; 293(3):464-76,_Fang, Anat Rec. 2011; 294(1):24-45).

Reg has been shown to be a motor neuron mitogen facilitating regeneration after injury and promoted neuronal survival, preserved neurite complexity and fasciculation, and protected contents from reactive oxygen species and prevented the formation of cortical and white matter lesions and reduced neuronal death and glial activation following toxic insults (Haldipur et al., Ann Clin Transl Neurol. 2014; 1(10):739-54). Kawahara found that Reg protein may protect neurons following seizures (Neuroscience. 2011:23; 175:273-80).

To date, specifically the shorter peptides and optimized Reg peptides and sequences described herein that are bioactive and highly homologous regions of human and mammalian Reg peptides that have been identified and patented by this inventor (U.S. Pat. Nos. 8,911,776; 8,816,047; 9,133,440; 7,989,415; 7,393,919; 9,321,812; 9,133,440; 8,829,158; 8,785,400; 8,383,578; 8,211,430; 7,989,412; 7,714,103; 7,393,919; 8,816,047; and 9,321,812), have not been considered as potential therapy for brain injury, stroke, Alzheimer's Disease, spinal cord injuries, and other central and peripheral nervous system conditions myocardial, renal or liver disease.

The bioactive region of the Reg receptor identified in this invention for which antibodies have been raised by this inventor, have not previously been described or considered for the development of stimulatory antibodies or small molecule therapy for binding to the Reg receptor, EXTL-3, resulting in new neurons, myocytes, hepatocytes and nephrons to be used in treatment of brain injury, stroke, spinal cord injuries, and other central and peripheral nervous system conditions, and for myocardial, renal and liver diseases.

This invention and sequences described include peptides contained within human Reg1a, Reg1b, Reg3a, Reg 4 and other mammalian peptide sequences for usage in the protection and generation of new neurons, including motor neurons, and myocardial cell protection and regeneration of cardiac myocytes during acute injury, which have not been described previously.

To date, none of the peptides described herein have been described in the prior art for usage in the generation of new neurons, myocytes or hepatocytes. While there is demonstration that the entire Reg gene protein improves spinal cord, myocardial and hepatic survival after organ injury. The usage of shorter Reg peptides, optimized Reg peptides, peptidomimetics to the Reg receptor and small molecules to the Reg receptor are novel to the field of regenerative medicine and have not been considered as potential therapy.

Formulations, derivatives, optimized forms of shorter Reg peptides previously described and patented by this inventor, and the identification of a 20-amino acid binding region on the EXTL-3/Reg receptor by this inventor along with peptidomimetics and stimulating antibodies to the Reg receptor have not previously been used in the treatment of stroke, spinal cord injury, peripheral neuropathy and other progressive diseases of the brain, heart or liver, all of which express receptors in times of organ injury.

These peptides described herein by this inventor have been used to transform progenitor cells in adult organs into new adult tissues in a given organ that is injured acutely via the Reg receptor (Levetan Endocr Pract. 2008; 14(9):1075-83). These human peptides including human Reg1a, Reg 3a, and Reg 4a have highly homologous regions to other mammalian Reg gene proteins and have been shown to interact with the Reg receptor, also known as EXTL-3. In acute injury, Reg gene proteins have been found to bind to Reg receptors in the heart, brain, and liver to generate new heart, brain and liver cells, and whereas this invention identifies the same bioactive binding arm of the mammalian and human Reg peptides that has been shown to interact with the Reg receptor have been shown by this inventor to transforming cells into functioning adult cells through the interaction Reg receptor. Reg receptors are found expressed in the brain, heart, pancreas, liver and other organs, thus targeted therapy in times of acute and chronic injury to these key organs can potentially prolong life and improve quality of life.

The use of Reg peptides, peptidomimetics, small molecules and antibodies targeting the Reg receptor and the 20-amino acid binding region (SEQ ID NO: 6), have not previously been described for the usage in ischemic brain injury, neuropathy, Alzheimer's disease, spinal cord injury, peripheral nerve disease and other central and peripheral nervous system diseases in which new neuron production could improve clinical status, nor in myocardial ischemia and myocardial disease and liver disease. These same bioactive and homologous regions of the Reg gene proteins in humans and other mammals can also be used for regeneration of neurons, myocytes, nephrons and hepatocytes.

This invention described herein is not contained in the prior art and consist of Reg peptide sequences and optimized peptide sequences including a 15-amino acid optimized mammalian Reg peptide (SEQ ID NO: 8), an optimized 14-amino acid human Reg peptide (SEQ ID NO: 12), a 7-amino acid human Reg peptide, an 8-amino acid human Reg and a 9-amino acid human Reg peptides that have been patented by this inventor (U.S. Pat. Nos. 8,911,776; 8,816,047; 9,133,440; 7,989,415; 7,393,919; 9,321,812; 9,133,440; 8,829,158; 8,785,400; 8,383,578; 8,211,430; 7,989,412; 7,714,103; 7,393,919; 8,816,047; and 9,321,812).

The peptides described in this invention interact with the Reg receptor, a cell surface receptor found expressed during the acute injury model in the brain, nephron, heart and liver and result in downstream generation of new cells producing new cardiac myocytes, new neurons, increased neuron length, new nephrons and new liver cells. This invention also describes a specific 20-amino acid binding region (SEQ ID NO: 6) within the Reg Receptor that is contained within its 919-amino acid Reg Receptor (SEQ ID NO: 7), which is the binding site within this invention. This invention also includes small molecules and stimulating antibodies to the 20-amino acid binding site within the 919-amino acid Reg receptor, also known as EXTL-3.

This invention also confirms that the Reg protein is found in the acute injury model of the brain, heart and liver and works through the Reg receptor. The peptides presented in this invention and the specific 20-amino acid binding region within this 919-amino acid human Reg Receptor (SEQ ID NO: 7), have not been described in the prior art for use in acute injury to the brain, spinal column, peripheral or central nervous system, for acute myocardial or liver injury.

This invention includes the generation of new neurons that are developed from formulations, derivatives, optimized forms including peptidomimetics of the peptides and stimulating antibodies to the Reg Receptor that are designed for the usage in the treatment of stroke, cerebral ischemia, spinal cord injury or other conditions of the central nervous system in which there is a deficit of neurons including conditions in which new neurons may improve mentation, or quality of life, with data demonstrating that intrathecal delivery may be effective in transection of the spinal cord.

Additionally, this invention includes formulations, derivatives, optimized forms including peptidomimetics of the peptides and stimulating antibodies to the Reg receptor that can improve cardiac function including acute myocardial ischemia, myocarditis, diminished ejection fraction, cardiomyopathies and other cardiac conditions in which improved myocyte mass can improve cardiac function and quality of life. Additionally, formulations, derivatives, optimized forms including peptidomimetics of the peptides and stimulating antibodies to the Reg receptor are used for acute and chronic liver injury in which new hepatocytes for which there are EXTL-3 receptors identified.

Routes of delivery of therapies described include, but are not limited to oral, intravenous, intra-arterial, subcutaneous delivery and intrathecal delivery and through the spinal canal/intrathecal for generation central neurons and for spinal cord injuries. Also, therapy may be given by organ specific targeting and may include direct administration liver via the umbilical and hepatic artery, femoral or radial arterial delivery to the heart or directly to the arteries supplying the heart, as is done in cardiac catheterizations, and intrathecal delivery and through the spinal canal for central nervous system as is done for antibiotic delivery for central nervous system infections and for chemotherapy delivery for central lesions.

### BACKGROUND OF THE INVENTION

With the advent of the Human Genome Project, we have been able to identify the genes expressed during an acute injury to the pancreas. As was shown in by many investigators, ligation of the pancreatic ducts, resulted in the formation of new islets. This inventor demonstrated that the human Reg gene peptides interact with EXTL-3, the Reg receptor. Others in the field have shown that progenitor cells are present in organs after embryogenesis can be transformed into adult cells.

For example, within the pancreatic ductal cell population (the extraislet exocrine tissue) which comprises 98% of the pancreas mass, there are progenitor cells that can give rise to new functional endocrine, insulin-producing islets within the human pancreas when there is acute injury (Inada, Proc Natl Acad Sci USA. 2008; 16; 105(50):19915-9., Davani, Stem Cells. 2007; 25(12):3215-22). Organs house progenitor cells, which can be transformed into functioning adult cells, as a protective mechanism, when there is acute injury to an organ.

The ability to upregulate genes such as the Reg genes, when acute injury occurs, results in the ability to protect the injured organ. This protective mechanism within each organ represents an ability of the body to respond to acute injury. Whether or not there is enough regeneration to overcome the injury, depends on the degree of the insult to the organ and the amount of regeneration. For example, the ability to form new neurons may or may not be enough to overcome destruction of neurons, in the case of ischemic injury to the brain or spinal cord injury.

Similarly, the rate of new myocardial cells may not be sufficient to negate ischemia to the cardiac myocytes that may occur as a result of rupture of a plaque within the vasculature of the myocardium.

Of significant importance in this invention, is that Reg genes are not expressed in the normal pancreas, brain or heart. It is not until, there is acute or chronic injury, that the Reg genes are found to be expressed.

Understanding of the Reg gene proteins and their receptor are part of the body's own protective mechanism of organ repair. The ability to accelerate neuron growth in the case of a stroke or spinal cord injury or myocardial regeneration to be sufficient to repair myocardial tissue would represent a true breakthrough in the field of regenerative medicine, especially in light of stroke and cardiac disease, are a major cause of mortality.

Cardiac disease is the number cause of death worldwide with 17.3 million deaths each year according to the World Health Organization In the US, 800,000 people die from cardiovascular disease or stroke. Stroke is the leading cause of serious long-term disability according to the Centers for Disease Control.

To date, shorter peptides and sequences, bioactive region of the Reg receptor and peptidomimetics and stimulatory antibodies to the Reg, described herein and patented by this inventor (U.S. Pat. Nos. 8,911,776; 8,816,047; 9,133,440; 7,989,415; 7,393,919; 9,321,812; 8,829,158; 8,785,400; 8,383,578; 8,211,430; 7,989,412; 7,714,103; 7,393,919; 8,816,047; and 9,321,812) have not been considered as potential therapy for brain injury, stroke, Alzheimer's Disease, other brain conditions and spinal cord injuries or acute or chronic myocardial, which is the invention herein.

The ability to generate fully functional adult cells, through the differentiation of progenitor cells has now been shown by many research groups among many organs via Reg proteins acting through the Reg receptor. Over the past several decades, the regenerating gene (Reg) family has emerged among many species, including humans, as a key initiating factor for regeneration of diverse organ systems. After fetal development when organs are populated with endocrine cells for the first time, the Reg genes and Reg gene proteins are usually undetectable but are upregulated in response to acute injury.

It is also known from the Human Genome Project that there are many genes that are responsible for the formation of cells of the different organs when organs are populated for the first time during fetal development (such as the brain, heart and pancreas) and such genes are expressed almost exclusively during embryological development and then only expressed when there is acute injury as a mechanism to help repair the injured organ.

This invention identifies and confirms peptide sequences that are highly conserved and 100% identical within the human Reg1a, human Reg1b, human Reg3a and human Reg 4 proteins and identical Reg peptide sequences within many other mammalian species. This inventor has previously described the role of 7-15-amino acid Human Reg peptides in pancreatic islet development. This present invention identifies peptide 7-15-amino acid sequences and optimized sequences of these peptides contained within the human Reg1a, Reg1b, Reg3a and Reg4 and more than a dozen other mammals that interact with the Reg Receptor for the formation of new neurons, cardiac myocytes, nephrons and liver cells.

This invention demonstrates the utility of these peptides, derivatives, peptidomimetics and stimulatory antibodies that have been generated from the specific binding regions of the Reg Receptor, EXTL-3. Others have provided evidence that Reg peptides play a direct role in stimulating new tissue. (Kapur R et al Islets. 2012; 4(1), Watanabe T et al., Proc Natl Acad Sci USA. 1994, 26; 91(9):3589-92. Zenilman M E, et al., Pancreas. 1998; 17:256-261.)

Previously, this inventor demonstrated that a human Reg protein has successfully been administered to human pancreatic ductal tissue devoid of islets resulted in a significant increase in insulin concentrations indicating new beta cell formation resulted a 3-fold rise in total beta cells staining insulin in STZ-rendered diabetic mice. (Levetan., et al, Endocr Pract. 2008; 14(9):1075-83.) This inventor has used human pancreatic ductal tissue to transform cells by the use of Reg peptides into new islets via the Reg receptor, which has been confirmed by others. (Li J, et al. Peptides 2009; 30:2242-9, Assouline-Thomas B G, Diabetes 2008, 57(Suppl; 1) A2413. Kapur R, et al, Islets. 2012; 4(1).)

The Section of Islet Cell and Regenerative Biology at Joslin Diabetes Center confirmed that the a 15-amino acid Reg was present in the newest beta cells and islets that were formed directly from branching proliferating extra-islet ducts, which also confirms that mechanism of action of Reg peptide is to form new beta cells from extra-islet exocrine tissue (Guo L et al, Diabetes. 2010, 59(suppl; 1) A2589). When Reg is inhibited by the administration of a blocking antibody in an animal model of pancreatic injury, there was attenuated recovery, also confirming that Reg's role is both protective and regenerative during acute pancreatic injury (Viterbo, et al. JOP. 2009; 10(1):15-23).

Similar to these findings, other data support that the Reg protein is an initiating factor to downstream regulation of new cells of the heart, liver, brain, spinal cord and peripheral neurons. For example, when Reg is initially expressed, PDX-1, PAX1, Ngn3,Nkx6.1, Sox9 and Ins, are not expressed, and once Reg is present, PDX-1, PAX1, Ngn3, Nkx6.1, Sox9 and Ins and other beta cell proliferation factors become present demonstrating that Reg activates downstream factors necessary for cell regeneration. (Vukkadapu S S Physiol Genomics 2005:21, 201-211, Kapur R., et al., Islets. 2012; 4(1).)

Also identified is a bioactive domain within the Reg receptor that is immunogenic and stimulatory antibodies to this binding site have been generated. A putative Reg receptor initially described in rodents, was found by this inventor to be present in human tissue. The prior art described by this inventor includes 14 and 15 optimized and native peptides interact with the Reg receptor and therefore can also be used for acute injury of the heart, brain and liver with downstream signaling resulting from the Reg peptides interacting with the Reg receptor, as can peptidomimetics and stimulatory antibodies to the bioactive 20-amino acid binding region within the 919-amino acid Reg Receptor/EXTL3. (Levetan C S., et al, Endocr Pract. 2008; 14(9):1075-83.)

This present invention demonstrates that 7-15-amino acid human and mammalian Reg and optimized Reg peptides identified by this inventor to be used for acute brain injury, spinal cord injury, stroke, conditions in which there is cerebral atrophy such as fronto-temporal atrophy, Alzheimer's Disease and other conditions in which new neurons and neuron outgrowth could be helpful and improve functional status and quality of life, along with cardiac conditions including cardiomyopathy and acute myocardial infarction, myocarditis and other conditions in which there is a diminished cardiac ejection fraction that can be improved with new cardiac myocytes.

The Reg receptor and has been described and known as hereditary Multiple Exostoses Gene Isolog with other names describing this receptor including the Reg Receptor, BOTV, BOTY, DKFZp686C2342, exostoses (multiple)-like 3, Exostosin-like 3, EXT-related protein 1, EXTL1, HHREG RECEPTOR, EXTR1, EXTL3 Glucuronyl-galactosylproteoglycan-4-alpha-N-acetylglucosaminyltransferase, KIAA0519, Multiple exostosis-like protein 3, REGR and RPR, exostoses (multiple-like 3, Glucuronyl-galactosyl-proteoglycan, 4-alpha-N-acetylglucosaminyltransferase, exostosin-like-3, Hereditary multiple exostoses gene isolog, reg receptor, Multiple exostosis-like protein 3, and EC2.4.1. The receptor was named for its similarities to the Exostoses family of genes by homology screening, but it was specifically noted that this receptor is not derived from the Exostoses (EXT and EXTL) genes. Rather, the Reg Receptor protein was categorized as a member of the Exostosin family because it demonstrates a 52% homology to the 262-amino acid C-terminal of the Exostosin-like 2 protein and a 40% homology with the 242-amino acid C-terminal of the Exostosin-like 1 protein, yet there is no homology of Reg Receptor to the N-terminal regions of Exostosin-1 or 2. (Kobayashi S. et al., Anat. Embryol. 207:11-15, 2003.)

This inventor demonstrated that peptides not contained within the human 14-amino acid Reg3a, are contained within the human Reg1a, Reg1b, Reg3a and Reg4 protein and bind to the Reg Receptor. Findings by this inventor demonstrate that the Reg receptor plays a key regulatory role in cell growth and generation of new cells from cells contained within organs. Further, the present invention identifies the binding region within human Reg1a, human Reg1b, human Reg3a and human Reg4 and a binding domain on Reg receptor, which are targets for the treatment of diabetes and other diseases for which there is need for new beta cells.

The present invention further demonstrates these 14 and 15-amino peptides, which have not been described in the prior art for generation of new neurons, hepatocytes or myocytes by binding to the Reg and are pivotal for the formation of new cells via direct usage of the peptide, derivatives, optimized versions, peptidomimetics that bind to Reg receptor or via stimulating antibodies generated from unique binding sites within the Reg receptor that generate new cardiac, nerve and liver cells.

This invention finds the Reg receptor to be a pivotal receptor and a specific site within the Reg receptor to be the site of Reg binding resulting in the translocation of the Reg receptor through the cytoplasm to the nucleus resulting in the generation of new cell growth, acceleration and turnover in the damaged organ. This invention demonstrates the ability to use shorter mammalian and human Reg peptides to interact with the Reg receptor resulting in downstream upregulation of transcription factors resulting in new brain neurons, cardiac myocytes, liver hepatocyte, when such organs are injured, when there is upregulation of the Reg proteins.

### BRIEF SUMMARY OF THE INVENTION

Mammalian Reg proteins have been shown to induce an upregulation of proteins involved in brain, peripheral and spinal cord neuron regeneration. The ability of the 15 amino-acid Reg peptide to improve nerve function and enhance regeneration in streptozotocin-induced diabetic mice was shown two decades ago as coincident to treatment of diabetic mice, yet clinical usage in man or conditions of peripheral nerve aberrations, is new to the art and not described as a potential useful treatment for diabetic neuropathy or other conditions of neuronal loss, nor was the concept that a shorter Reg peptides or optimized Reg peptides considered by, this inventor or others in the art as potentially useful for regeneration of neurons in diabetes, brain or spinal cord neurons, nor has there been prior consideration in the art for usage of Reg peptides, optimized Reg peptides, peptidomimetics, small molecules or stimulating antibodies to generate new cells in injured organs that possess the Reg receptor.

Embodiments of the present invention provide for novel agents and methods for neuron, cardiac myocyte, and liver cell regeneration agents that have not previously been described using optimized mammalian and human Reg peptides, peptidomimetics, stimulatory antibodies and small molecules to interact with the Reg receptor in organs expressing the Reg receptor to generate new cells following organ injury. This inventor describes in vitro and in vivo in human tissue cell and mammals demonstrating that Reg proteins interact with the Reg receptor for the regeneration of new cells from progenitor cells in organs postnatally. This inventor demonstrates how these studies are translatable to humans and how shorter Reg peptides, optimized Reg peptides, peptidomimetics to the Reg receptor, stimulatory antibodies to the Reg receptor and how small molecules developed to the Reg receptor trigger downstream generation of new cells to tissue and organs expressing the Reg receptor when there is organ damage.

Therapies include homologous human 7-15-amino acid peptide sequences within the Reg1a, Reg1b, Reg3a and human Reg4 and a 15-amino acid optimized mammalian Reg peptides, as well as, stimulatory antibodies generated from a unique 20-amino acid binding region within the 919-amino acid protein human Reg receptor for the usage of new neurons, cardiac myocytes, and hepatocytes. This invention includes formulations, derivatives, optimized forms of human peptides described, and also includes peptidomimetics and stimulatory antibodies serving as peptidomimetics that are designed for the usage in the treatment of conditions of acute and chronic organ injury in which Reg gene peptide helps generate new adult cells within the adult organ suffering an insult.

This invention includes methods for cardiac, neuron, or hepatocyte generation within the organ in which there has been injury which expresses the Reg receptor for treatment of patients with heart disease, myocardial infarction, stroke, spinal cord injury, peripheral neuropathy, and liver disease from many causes including toxins, tumors, infections and other conditions in organs requiring generation of new cells. This invention includes methods for direct delivery of agents specified in this invention for generation of new adult neurons, liver cells within a damaged brain, spinal cord, heart, liver and provides to patients via direct delivery via arterial delivery via radial or femoral artery to the heart and arterial targeting to liver via injection through the umbilical and portal vessels and intrathecal delivery or within the spinal column for patients with stroke and spinal cord injuries, but may also include targeted oral and intravenous, subcutaneous delivery with organ specific targeting and may include direct administration to the liver, brain and heart. SEQ ID NOs: 1, 2, 3, 4, 5, 8-27 may be delivered as a peptide or encapsulated as a nanoparticle and delivered via oral delivery, oral targeted delivery to the liver, heart, brain, spinal cord or delivered via intravenous, intra-arterial, subcutaneously, intrathecally or directly to the injured organ either as a peptide or nanoparticle.

This inventor identified EXTL-3 as the Reg receptor in man (Levetan Endocr Pract. 2008; 14(9):1075-83). This inventor has also identified a 20-amino acid binding region on the 919-amino acid EXTL-3 Reg receptor from which stimulatory antibodies, monoclonal antibodies, small molecules developed from SEQ ID NO: 6, which result in the formation of new neurons, hepatocytes, myocytes, and renal cells may be may be targeted or delivered directly or indirectly and may be encapsulated as a nanoparticle and delivered via oral delivery, oral targeted delivery to the liver, heart, brain, spinal cord, or delivered via intravenous, intra-arterial, subcutaneously, intrathecally or delivered directly to the injured organ to the injured organ.

In the first embodiment, the present invention provides for the discovery of specific regenerative bioactive peptide sequences within the human Reg protein that are referred to as embodiments of regenerative peptides including the discovery of the following bioactive sequences and their optimized versions which include, but are not limited to the following 7-15-amino acid sequences and optimized sequences within the human and mammalian Reg peptides:

This is the 15-amino acid peptide found within the mammalian Reg proteins.

(SEQ ID NO: 1)
Ile-Gly-Leu-His-Asp-Pro-Ser-His-Gly-Thr-Leu-Pro-
Asn-Gly-Ser
(SEQ ID NO: 1)
IGLHDPSHGTLPNGS

This is the 14-amino acid peptide found within the human Reg proteins.

(SEQ ID NO: 2)
Ile-Gly-Leu-His-Asp-Pro-Thr-Gln-Gly-Thr-Glu-Pro-
Asn-Gly
(SEQ ID NO: 2)
IGLHDPTQGTEPNG

This is the 7-amino acid peptide found within the mammalian and human Reg proteins.

(SEQ ID NO: 3)
Trp-Ile-Gly-Leu-His-Asp-Pro
(SEQ ID NO: 3)
WIGLHDP

This is the 8-amino acid peptide found within the human and mammalian Reg proteins.

(SEQ ID NO: 4)
Val-Trp-Ile-Gly-Leu-His-Asp-Pro
(SEQ ID NO: 4)
VWIGLHDP

This is the 9-amino acid peptide found within the human and mammalian Reg proteins.

(SEQ ID NO: 5)
Asn-Val-Trp-Ile-Gly-Leu-His-Asp-Pro
(SEQ ID NO: 5)
NVWIGLHDP

In the second embodiment, this discovery includes a 20-amino acid human specific binding site on the Reg Receptor, EXTL-3, with 100% homology to 17 other mammals (SEQ ID NO: 6) within the 919-amino acid Reg Receptor (SEQ ID NO: 7) from which a stimulatory antibody to the Reg Receptor have been generated from SEQ ID NO: 6)

This is a 20-amino acid peptide found within the human Reg receptor. EXTL-3.

(SEQ ID NO: 6)
Cys-Lys-Lys-Ser-Ile-Glu-Asn-Ala-Lys-Gln-Asp-Leu-
Leu-Gln-Leu-Lys-Asn-Val-Ile-Ser
(SEQ ID NO: 6)
CKKSIENAKQDLLQLKNVIS

This is the 919-amino acid human Reg receptor, also known as EXTL-3

(SEQ ID NO: 7)
MTGYTMLRNGGAGNGGQTCMLRWSNRIRLTWLSFTLFVILVFFPLIAH
YYLTTLDEADEAGKRIFGPRVGNELCEVKHVLDLCRIRESVSEELLQL
EAKRQELNSEIAKLNLKIEACKKSIENAKQDLLQLKNVISQTEHSYKE
LMAQNQPKLSLPIRLLPEKDDAGLPPPKATRGCRLHNCFDYSRCPLTS
GFPVYVYDSDQFVFGSYLDPLVKQAFQATARANVYVTENADIACLYVI
LVGEMQEPVVLRPAELEKQLYSLPHWRTDGHNHVIINLSRKSDTQNLL
YNVSTGRAMVAQSTFYTVQYRPGFDLVVSPLVHAMSEPNFMEIPPQVP
VKRKYLFTFQGEKIESLRSSLQEARSFEEEMEGDPPADYDDRIIATLK
AVQDSKLDQVLVEFTCKNQPKPSLPTEWALCGEREDRLELLKLSTFAL
IITPGDPRLVISSGCATRLFEALEVGAVPVVLGEQVQLPYQDMLQWNE
AALVVPKPRVTEVHFLLRSLSDSDLLAMRRQGRFLWETYFSTADSIFN
TVLAMIRTRIQIPAAPIREEAAAEIPHRSGKAAGTDPNMADNGDLDLG
PVETEPPYASPRYLRNFTLTVTDFYRSWNCAPGPFHLFPHTPFDPVLP
SEAKFLGSGTGFRPIGGGAGGSGKEFQAALGGNVPREQFTVVMLTYER
EEVLMNSLERLNGLPYLNKVVVVWNSPKLPSEDLLWPDIGVPIMVVRT
EKNSLNNRFLPWNEIETEAILSIDDDAHLRHDEIMFGFRVWREARDRI
VGFPGRYHAWDIPHQSWLYNSNYSCELSMVLTGAAFFHKYYAYLYSYV
MPQAIRDMVDEYINCEDIAMNFLVSHITRKPPIKVTSRWTFRCPGCPQ
ALSHDDSHFHERHKCINFFVKVYGYMPLLYTQFRVDSVLFKTRLPRDK
TKCFKFI

In a third embodiment, this discovery also includes “Optimized Regenerative Peptides” including peptidomimetics which refers to variations of Regenerative Peptides wherein the peptide has been modified to increase the stability, solubility, including formulations that have increased protease resistance, reduced immunogenicity, Tmax and bioavailability compared to the native peptide and/or provide greater ease in administration. In certain aspects, the modifications can include acetylation, amidation, pegylation, and/or cyclization, or any combinations of these.

This is the optimization of the mammalian 15-amino acid Reg peptide by blocking with an n-terminal acetyl group and a c-terminal amide group.

(SEQ ID NO: 8)
Ac--Ile-Gly-Leu-His-Asp-Pro-Ser-His-Gly-Thr-Leu-
Pro-Asn-Gly-Ser-NH2

This is a peglyated version of the mammalian 15-amino acid Reg peptide

(SEQ ID NO: 9)
Ac--Ile-Gly-Leu-His-Asp-Pro-Ser-His-Gly-Thr-Leu-
Pro-Asn-Gly-Ser--(Peg-40 KD-maleimide)-NH2

This is a peglyated version of the mammalian 15-amino acid Reg peptide Peg-40KD-maleimide-3-Mpa-Ile-Gly-Leu-His-Asp-Pro-Ser-His-Gly-Thr-Leu-Pro-Asn-Gly-Ser-NH2 (3-Mpa=3-mercaptopropionic acid) (SEQ ID NO: 10)

This is a cyclized version of the mammalian 15-amino acid Reg peptide to include but is not limited to:

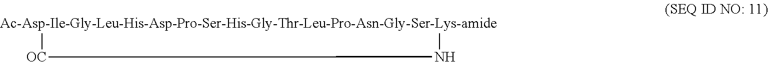

This is the optimization of the 14-amino acid human Reg peptide by blocking with an n-terminal acetyl group and a c-terminal amide group.

(SEQ ID NO: 12)
Ac--Ile-Gly-Leu-His-Asp-Pro-Thr-Gln-Gly-Thr-Glu-
Pro-Asn-Gly--NH2

This is a peglyated version of the human 14-amino acid Reg peptide

(SEQ ID NO: 13)
Ac--Ile-Gly-Leu-His-Asp-Pro-Thr-Gln-Gly-Thr-Glu-
Pro-Asn-Gly--(Peg-40 KD-maleimide)-NH2

This is a peglyated version of the mammalian 14-amino acid peptide

(SEQ ID NO: 14)
Peg-40 KD-maleimide-3-Mpa-Ile-Gly-Leu-His-Asp-
Pro-Thr-Gln-Gly-Thr-Glu-Pro-Asn-Gly--NH2
(3-Mpa = 3-mercaptopropionic acid)

This is the cyclized versions of the human 14-amino acid Reg peptide to include but are not limited to:

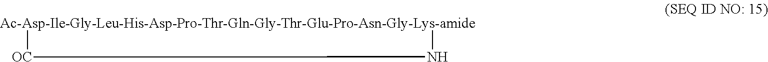

This is the optimization of the 7-amino acid human and mammalian Reg peptide by blocking with an n-terminal acetyl group and a c-terminal amide group.

(SEQ ID NO: 16)
Ac--Trp-Ile-Gly-Leu-His-Asp-Pro--NH2

This is a peglyated version of the human and mammalian 7-amino acid Reg peptide.

(SEQ ID NO: 17)
Ac-Trp-Ile-Gly-Leu-His-Asp-Pro(Peg-40KD-
maleimide)-NH2

This is a peglyated version of the mammalian and human 7-amino acid Reg peptide.

(SEQ ID NO: 18)
Peg-40KD-maleimide-3-Mpa-Trp-Ile-Gly-Leu-His-Asp-
Pro-NH2(3-Mpa = 3-mercaptopropionic acid)

Cyclized versions of the human 7-amino acid human and mammalian Reg peptide to include but are not limited to:

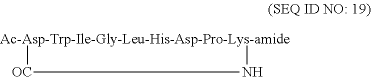

This is the optimization of the 8-amino acid human and mammalian Reg peptide by blocking with an n-terminal acetyl group and a c-terminal amide group.

(SEQ ID NO: 20)
Ac-Val-Trp-Ile-Gly-Leu-His-Asp-Pro--NH2

This is a peglyated version of the human and mammalian 8-amino acid Reg peptide.

(SEQ ID NO: 21)
Ac-Val-Trp-Ile-Gly-Leu-His-Asp-Pro(Peg-40KD-
maleimide)-NH2

This is a peglyated version of the human and mammalian 8-amino acid Reg peptide

(SEQ ID NO: 22)
Peg-40KD-maleimide-3-Mpa-Val-Trp-Ile-Gly-Leu-His-
Asp-Pro-NH2 (3-Mpa = 3-mercaptopropionic acid)

Cyclized versions of the human and mammalian 8-amino acid Reg peptide to include but are not limited to:

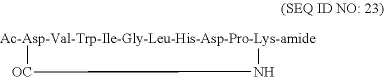

This is the optimization of the 9-amino acid human and mammalian Reg peptide by blocking with an n-terminal acetyl group and a c-terminal amide group.

(SEQ ID NO: 24)
Ac-An-Val-Trp-Ile-Gly-Leu-His-Asp-Pro--NH2

This is a peglyated version of the human and mammalian 9-amino acid Reg peptide

(SEQ ID NO: 25)
Ac-Asn-Val-Trp-Ile-Gly-Leu-His-Asp-Pro(Peg-40KD-
maleimide)-NH2

This is a peglyated version of the human and mammalian 9-amino acid Reg peptide

(SEQ ID NO: 26)
Peg-40KD-maleimide-3-Mpa-Asn-Val-Trp-Ile-Gly-Leu-
His-Asp-Pro-NH2 (3-Mpa = 3-mercaptopropionic acid)

Cyclized versions of the human and mammalian 9-amino acid Reg peptide to include but are not limited to:

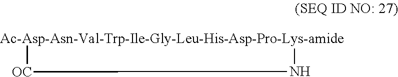

In a fourth embodiment, the in vivo generation of new neurons, cardiac myocytes, nephrons and hepatocytes occurs from the administration of peptidomimetics and includes formulations, derivatives, optimized forms and also include peptides and peptidomimetic agents that bind to the human Reg Receptor and a specific binding region within the Reg Receptor are presented in this invention with the modality of delivery including, but is not limited to: oral, intravenous, subcutaneously, intra-arterial, intrathecal and into the spinal column and targeted or delivered directly or indirectly to the brain, spinal column, heart and liver.

In a fifth embodiment, this inventions provides new therapy and includes specific methodology and timing for administration of Regenerative peptides, optimized regenerative peptides, regenerative peptidomimetics and stimulatory antibodies to the Reg receptor and specific methods for generation of new cells for usage, but not limited to generation of neurons for use in stroke, Alzheimer's disease, ischemic brain disease, and other disease of the brain that may be aided by more neurons including fronto-temporal atrophy and other neurological conditions that will be aided and improve quality of life by productions of new neurons; cardiac diseases including acute myocardial infarction, myocarditis, dilated cardiomyopathy and conditions in which additional myocytes will improve cardiac function, reduce congestive heart failure, and improve quality of life for patients with cardiovascular disease; new hepatocytes for usage in patients with liver disease by infection, toxin or hepatic surgery.

In a sixth embodiment, an innovative therapy for accelerated generation of the ex-vivo generation of beta cells by methodology for administration of regenerative peptides and/or formulations, derivatives, optimized forms including peptidomimetics and stimulatory antibodies to the Reg receptor that are formed ex-vivo using progenitor cells or tissue from organs expressing Reg receptors and regenerative peptide formulations, derivatives, optimized forms including peptidomimetics and stimulating Reg receptor antibodies for ex-vivo transformation of progenitor cells or tissue from organs expressing Reg receptors from to new neurons, new cardiac myocytes and new hepatocytes from adult human tissue of the brain, heart or liver, and that new adult cells may be administered intravenously, subcutaneously, intra-arterial, intrathecal delivery and delivery into the spinal column and delivery including delivery directly or indirectly to the brain, liver, spinal column and heart that are appropriate targets to optimize efficacy.

In a seventh embodiment, the present invention provide pharmaceutical formulations and unit dose forms of Reg peptides alone or in combination with one or more other active pharmaceutical ingredients (APIs). In one embodiment, the API is an agent in soluble liposome or nanoparticle preparations that allow Reg peptides to be administered by a variety of routes, including subcutaneously, intramuscularly, intravenously, intra-arterially, and even orally, depending on the formulation selected. In one embodiment, the formulation comprises a targeting agent for targeted administration to specific locations, receptors, cells, tissues, organs, or organ systems including targeting to the liver, brain, spinal column or heart.

In eighth embodiment, this invention provides new therapy and specific methods for the transformation of new neurons, new cardiac myocytes and new hepatocytes from adult tissue of the brain, heart or liver.

### BRIEF DESCRIPTION OF THE DRAWINGS

FIG. 1 demonstrates the expression of organs in man expressing the Reg receptor https://www.proteinatlas.org/ENSG00000012232-EXTL3/tissue

FIGS. 2A-C are graphics depicting the 3-dimensional structures of human Reg1a, human Reg3a and Hamster Reg 3gamma by Swiss-Prot folding algorithms with the Reg peptides contained within the Red circled binding arm. The Reg peptides are contained within the Red circled binding arm.

FIG. 3 is a set of sequences which demonstrate peptide homology between human Reg1a, Reg1b, Reg3a and Reg 4 and the Hamster Reg3gamma. The common sequences are highlighted in red.

FIG. 4 is the 919-amino acid sequence of the Reg Receptor with the 20-amino acid binding domain (SEQ ID NO: 6) presented in this invention is bolded.

FIG. 5 demonstrates the results of studies generating stimulatory antibodies to a 20-amino acid binding site within the Reg Receptor.

FIG. 6 provides the protocol for development of stimulatory antibodies to the binding domains within the Reg receptor

FIG. 7 is the documentation of titer controls and norms for the antibody studies.

FIGS. 8A-C are images of an SDS PAGE gel and Western blot results which demonstrates the results from studies conducted to demonstrate Reg Receptor expression and purification utilizing 293T cells that were transfected with Reg Receptor expression plasmid DNA. FIG. 8A demonstrates the use of sodium dodecyl sulfate polyacrylamide gel electrophoresis (SDS-PAGE) to illustrate the high purity of the Reg Receptor. FIGS. 8B and 8C are the Western blot results showing that the purified protein is the Reg Receptor using the antibody to the FLAG epitope (FIG. 8B) and Reg Receptor (CD104) (FIG. 8C).

FIG. 9 is a graph which shows the 7-amino acid Reg peptide, the 8-amino acid Reg peptide and the 9-amino acid Reg peptide all bind directly to Reg Receptor.

FIG. 10 is an image of a Western Biol which demonstrates a confirmation study to evaluate the translocation of the Reg Receptor from the cytoplasmic membrane of human extra-islet exocrine tissue. The Western blot analysis identified he presence of the Reg receptor on the cytoplasmic membrane and the movement of the Reg Receptor from the cytoplasmic membrane through the cytoplasm to the nucleus in the presence of the Regenerative peptides 7aa (SEQ ID NO: 3), Baa (SEQ ID NO: 4) and 9aa (SEQ ID NO: 5).

FIG. 11 is a table which demonstrates the results showing upregulated transcription factors stimulated by the Regenerative peptides acting through their receptor in an in-vivo mouse study of injected with peptides SEQ ID NO: 4, SEQ ID NO:5, SEQ ID NO: 8, SEQ ID NO:1 and SEQ ID NO:16

FIG. 12 is a set of sequences which demonstrate the 7-amino acid human Reg sequences (SEQ ID NO: 3) that has 100% homology with Reg sequences found in other mammals including chimpanzee, rat, mouse, golden hamster, guinea pig, rabbit, pig, sheep, cow, white-cheeked gibbon, Sumatran orangutan, Lowland gorilla, white-tufted-eared marmoset, European domestic ferret, which have not been described in the prior art as being a peptide which has not been described in the prior art as being a peptide that interacts with the Reg receptor for the generation of new neurons, cardiac myocytes, nephrons and hepatocytes

FIG. 13 is a set of sequence which demonstrate the 8-amino acid human Reg sequence (SEQ ID NO: 4) that has 100% homology with Reg sequences found in other mammals including chimpanzee, rat, golden hamster, guinea pig, rabbit, pig, sheep, cow, white-cheeked gibbon, Sumatran orangutan, Lowland gorilla, white-tufted-eared marmoset, which have not been described in the prior art as being a peptide that interacts with the Reg receptor that can generate for generation of as a peptide for generation of new neurons, cardiac myocytes, nephrons and hepatocytes.

FIG. 14 is a set of sequence which demonstrate the 9-amino acid human Reg sequences (SEQ ID NO: 14) that has 100% homology with Reg sequences found in other mammals including chimpanzee, rat, mouse, golden hamster, white-cheeked gibbon, Lowland gorilla, and white-tufted-eared marmoset, which have not been described in the prior art as being a peptide that interacts with the Reg receptor that can generate for generation of being a peptide for generation of new neurons, cardiac myocytes, nephrons and hepatocytes.

FIG. 15 is a set of sequence which demonstrates the 20-amino acid human sequence has been identified within the Reg receptor that has 100% homology with Reg receptor sequences found in other mammals including chimp, rat, mouse, guinea pig, rabbit, dog, cow, opossum, galago, white-cheeked gibbon, Sumatran orangutan, macaque, Lowland gorilla, white-tufted eared marmoset, horse and Tasmanian devil from which stimulatory antibodies and small molecules can be generated to interact with this receptor site resulting in new neurons, hepatocytes and myocytes

FIGS. 16A and B are microscopic images which show immunofluorescent staining of Reg receptor on the cell surface of human pancreatic exocrine ductal cells. In utilizing Cy3 immunofluorescent staining of Reg receptor in human pancreatic ductal cells in standard medium, there is immunofluorescent staining of Reg receptor, which is well-defined at the cell borders indicating surface expression of Reg receptor on the cytoplasmic membrane of cells (FIG. 16A). FIG. 16B demonstrates the difference in Reg receptor staining when cells are exposed to Reg.

FIG. 17 shows the protocol for the stability testing in human plasma for the Reg peptides SEQ ID NO: 1, SEQ ID NO: 8, SEQ ID NO:3 and SEQ ID NO:16

FIG. 18 is a table which shows the Reg peptides SEQ ID NO: 1, SEQ ID NO: 8, SEQ ID NO: 3, SEQ ID NO:16 and the incubation time points used in the stability testing in human plasma.

FIG. 19A is a table which shows the results of the Regenerative Peptides SEQ ID NO:1 incubated in human plasma.

FIG. 19B is a table which shows the results of the Reg peptides SEQ ID NO: 8 incubated in human plasma.

FIG. 19C is a graph which shows the results of the Regenerative Peptides SEQ ID NO:1 and SEQ ID NO: 8 incubated in human plasma. SEQ ID NO: 8 is significantly more resistant to proteolysis in human plasma compared to SEQ ID NO: 1

FIG. 20A is a table which shows the results of the Regenerative Peptides SEQ ID NO:16 incubated in human plasma.

FIG. 20B is a table which shows the results of the Regenerative Peptides SEQ ID NO:3 incubated in human plasma.

FIG. 20C is a graph which shows the results of the Regenerative Peptides SEQ ID NO: 16 and SEQ ID NO: 3 incubated in human plasma. SEQ ID NO: 16 is significantly more resistant to proteolysis in human plasma compared to SEQ ID NO:3

### DETAILED DESCRIPTION OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Over the past decade, the regenerating gene (Reg or REG) family has emerged among many species, including humans, as a potential key initiating factor in the process of new cell formation within an organ following acute injury, including the brain, heart and liver. Although Reg is typically expressed during embryogenesis when organs are being populated for the first time and downregulated after fetal development, the Reg genes are upregulated within the brain, pancreas, heart and liver when there is acute injury with the formation of new neurons, islets, cardiac myocyte, renal nephron and hepatocyte neogenesis within the injured organs (Levetan. J Diabetes. 2010 June; 2(2):76-84, Levetan Endocr Pract. 2008; 14(9). After fetal development, the Reg genes are usually undetectable, but are upregulated in response to acute organ injury.

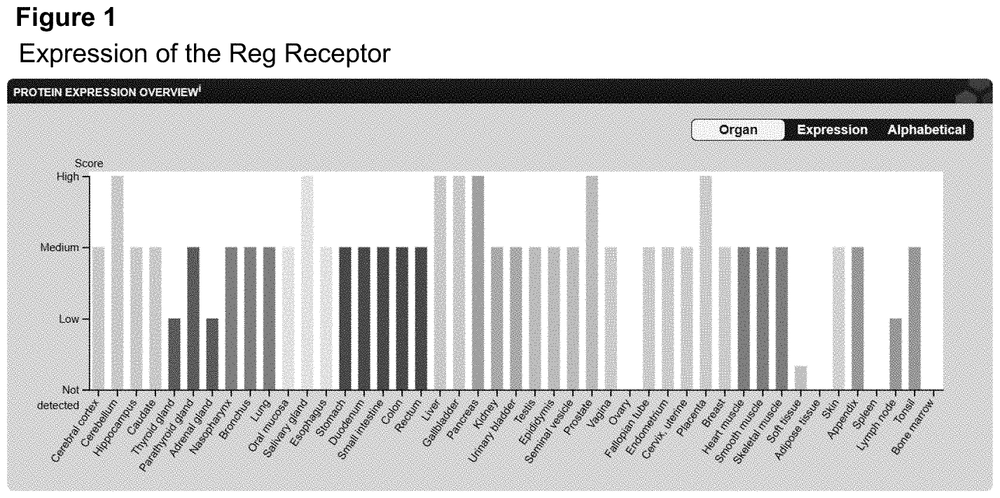

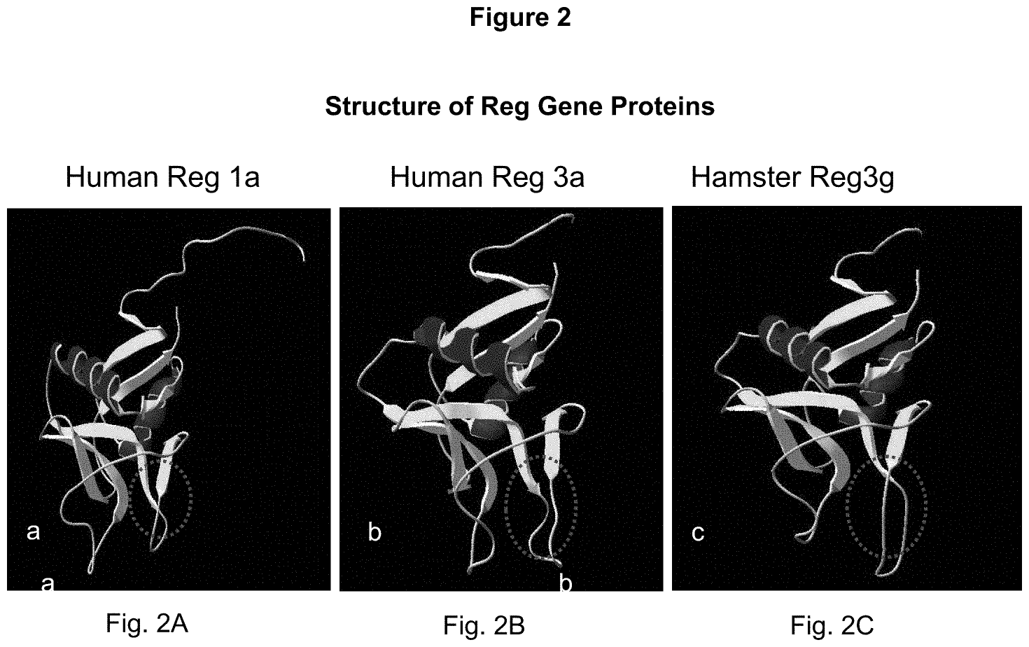

FIG. 1 A demonstrate the organs in man expressing the Receptor. FIG. 2A-C are illustrations showing the similarities and differences between three dimensional structures of human Reg1a, Reg3a and the Hamster Reg3 gamma peptides based on their primary-amino acid sequences and by SwissProt folding algorithms. FIG. 2A shows the three-dimensional structure for Human Reg1a. FIG. 2B shows the three-dimensional structure for Reg3a. FIG. 2C shows the three-dimensional structure for the hamster Reg3gamma. The circled region of the protein that contains the Regenerative peptides described in this invention, which bind to Reg Receptor. Red circled arm indicates the bioactive region and homologous sequences of the Reg proteins that are the binding arm to the Reg receptor.

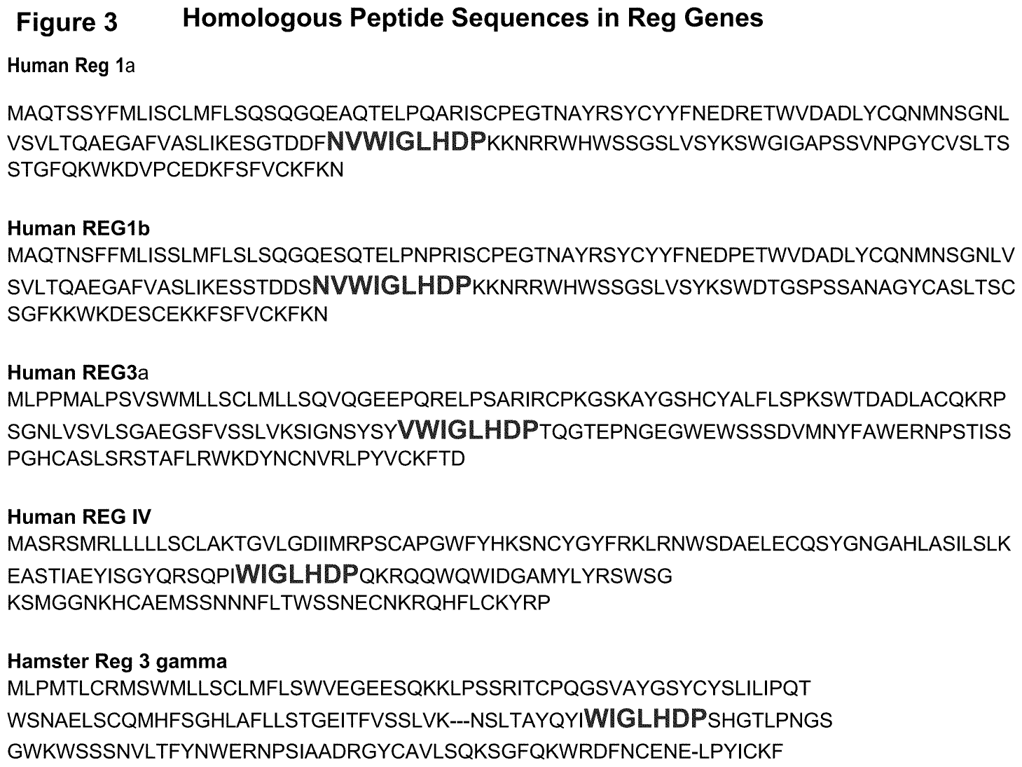

FIG. 3 is an alignment of the 166-amino acid sequence for Reg1a, the 166-amino acid sequence for Reg1b, the 175-amino acid sequence for Reg3 alpha, the 158-amino acid sequence for Reg4, and the mammalian Reg3gamma protein have been shown in the prior art to interact with the Reg receptor, resulting in downstream generation of new adult cells. FIG. 3 shows the identification of the 100% homologous sequence within the human Reg1a, Reg1b, Reg3a, Reg4 and the Hamster Reg3gamma peptides that is located within the binding arm of the protein that binds to the Reg Receptor. This exact sequence is also found in the human, chimpanzee, rat, mouse, golden hamster, guinea pig, rabbit, pig, sheep, cow, white-cheeked gibbon, Sumatran orangutan, Lowland gorilla, white-tufted-eared marmoset, and European domestic ferret.

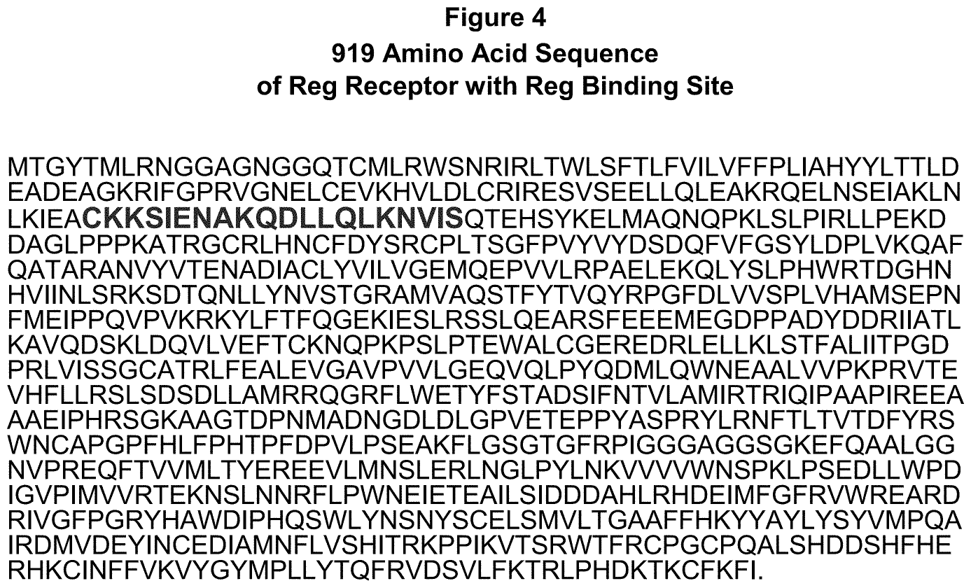

FIG. 4 shows the 919-amino acid Reg Receptor (SEQ ID NO: 7) and the 20-amino acid domain (SEQ ID NO: 6) in red that was identified in this discovery that is a binding domain for the Regenerative Peptides, and from which stimulatory antibodies to this region of the receptor have been generated.

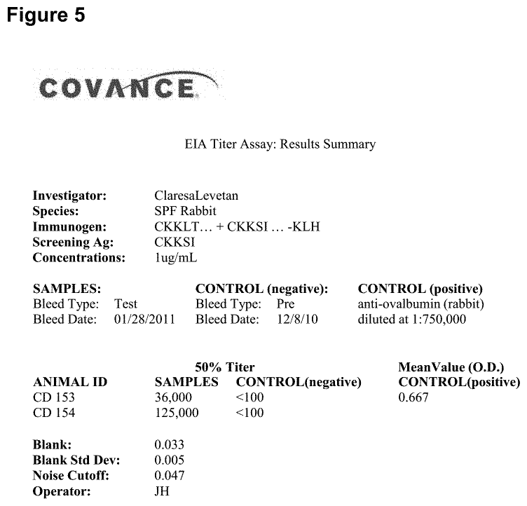

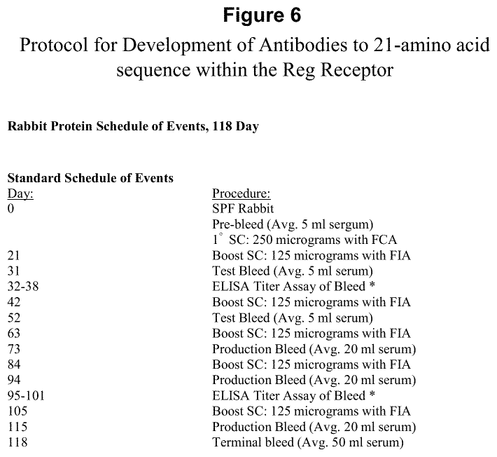

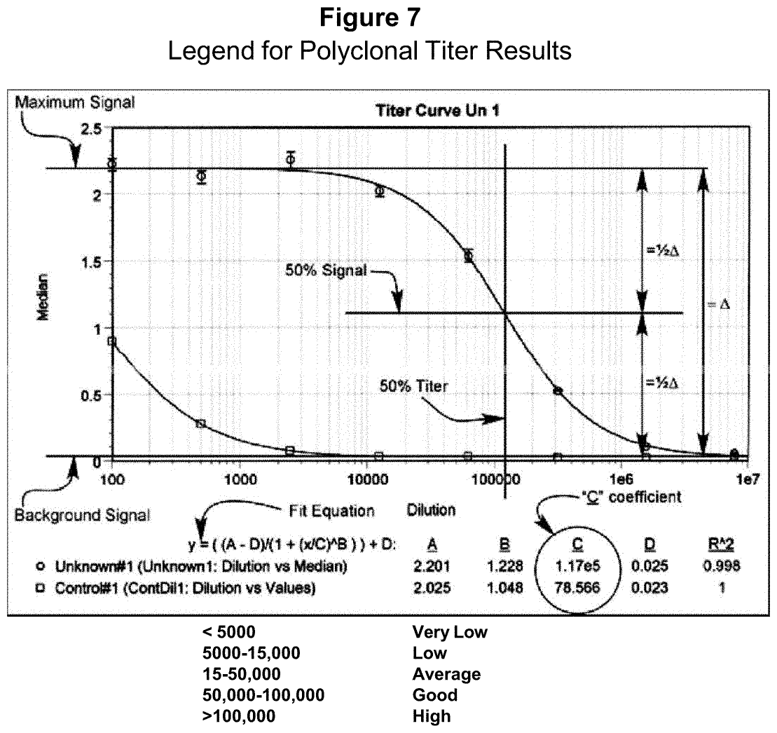

FIGS. 5-7 were from studies undertaken by this inventor to develop stimulatory antibodies to the 20-amino acid bioactive region of the 919-amino acid Reg Receptor/EXTL-3 to accelerate the downstream regulation of transcription factors to develop new cells. progression of beta cell formation by stimulating potential binding sites on the Reg Receptor. For the production of in stimulatory antibodies and identification and confirmation of the 20-amino acid binding region of EXTL-3, many sequences were evaluated within the N-terminal portion of the Reg receptor/Receptor within amino acids 1-332 of (SEQ ID NO:7) to find the region to which antibodies were produced, which this inventor identifies as the binding domain of the Reg peptides. Consistently, in Enzyme ImmunoAssay studies measuring titers from peptide sequences within SEQ ID NO:7, resulted in very high polyclonal antibodies being raised to a 20-amino acid Reg receptor sequence (SEQ ID NO: 8) (amino acids 117-136). The results of the Enzyme Immunoassay are summarized in FIG. 5. FIG. 6 demonstrates the standard protocol used for development of antibodies to peptide regions within Reg receptor. Data sets were taken from the bleed after the day 0 and day 21 boosts. The animals were injected with a peptide of SEQ ID NO: 6 and were conjugated to keyhole limpet hemocyanin (KLH). The screening antigen is not conjugated to KLH so that the response solely to the peptide and not to KLH can be identified. The 50% titer is a dilution value where the signal is half-way between the peak and the baseline, so the higher the dilution value (titer), the greater the response to the antigen. The positive control is an internal control that was generated from ovalbumin antibodies in rabbit. At a dilution of 1:750,000, the absorbance fell within a range of 0.45 to 0.9. In the case of the response to SEQ ID NO: 6, there was a high response (FIG. 5). The test bleed taken 31 days after the day 0 and 21 day boosts for CD 153 showed a 50% titer of 36,000 which is an average response, and CD 154 showed a 50% titer of 125,000 which is a high response according to the titer reference range shown in FIG. 7. Studies are underway to evaluate the efficacy of the antibody generated with and without the presence of Reg peptide demonstrating that the antibodies to the Reg peptide interaction with Reg receptor to generate new neurons, cardiac myocytes, nephrons and hepatocytes.

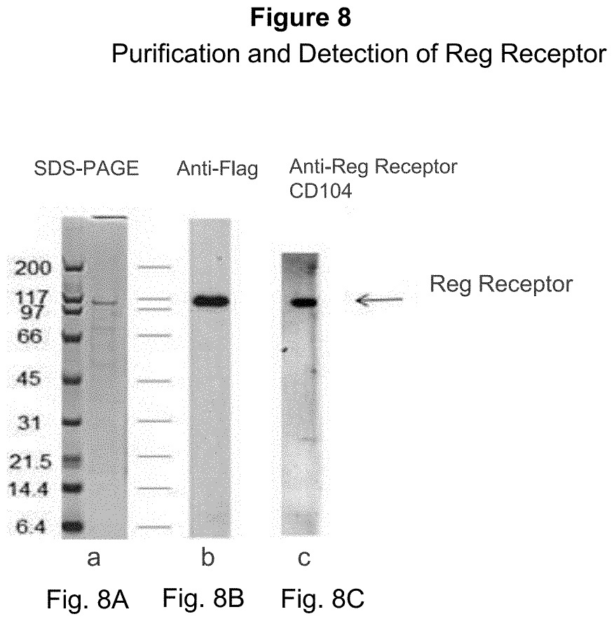

FIGS. 8A-C demonstrates the results from studies conducted to demonstrate Reg Receptor expression and purification utilizing 293T cells that were transfected with Reg Receptor expression plasmid DNA. Cells were collected after 72 hours. The Reg Receptor was tagged with FLAG epitope and FLAG resin was utilized to purify out the Reg Receptor. As shown in FIG. 7C, the Reg receptor was highly purified. The Reg receptor was purified by Anti-Flag M2 affinity gel. Target protein was confirmed by 4-12% SDS-PAGE and Western-blot. FIG. 8A demonstrates the use of sodium dodecyl sulfate polyacrylamide gel electrophoresis (SDS-PAGE) to illustrate the high purity of the Reg Receptor. FIGS. 8B and 7C are the Western blot results showing that the purified protein is the Reg Receptor using the antibody to the Reg Receptor (CD104). The Reg Receptor is shown in FIG. 8C to be highly purified. To then demonstrate the direct binding of Reg peptides to the Reg receptor, the purified Reg receptor was coated onto 96 well plate by using bicarbonate coating buffer, pH 9.6; 4° C. overnight at concentration 3 ug/ml, 100 ul per well. Plates were coated overnight coated plate and washed three times with 0.5×TBST and blocked with 3% BSA and rotated at room temperature for 1 hour. After blocking, plates were washed three times with 0.5×TBST. Peptides were then diluted with TBST buffer and added into wells in duplicate then left to bind at room temperature for 1 hour. After washing three times, 100 ng/ml strep-HRP was added into plate at 100 ul/well and rotated at room temperature for one hour. AB ST reagents were warmed to room temperature, mixed immediately before using. Then 100 ul was added to each well and read after 25 minutes reaction and absorbance was evaluated at 405 nm by a Spectramax M5 plate reader. The purified Reg Receptor was coated on plates. Then plates were blocked with BSA solution. Subsequently, the Reg peptides were added into the wells, and HRP-straptavidin and its substrates were added into the wells to reveal the interaction between Receptor and the peptide.

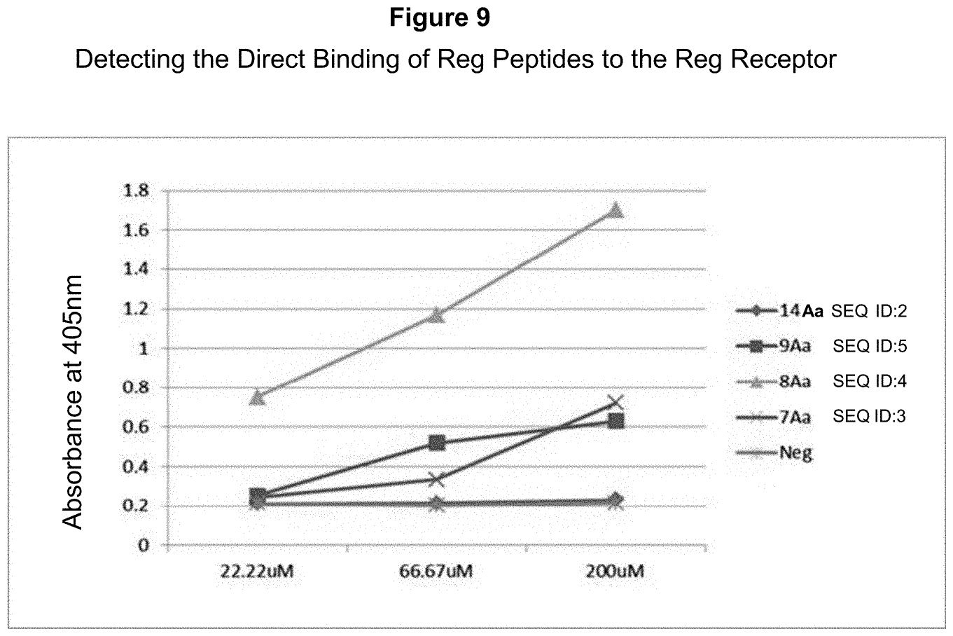

FIG. 9 shows the 7-amino acid Reg peptide (SEQ ID NO: 3), the 8-amino acid Reg peptide (SEQ ID NO: 4) and the 9-amino acid Reg peptide all show binding (SEQ ID NO: 5) to the Reg Receptor. The scrambled control peptide did not bind to Reg Receptor (FIG. 8).

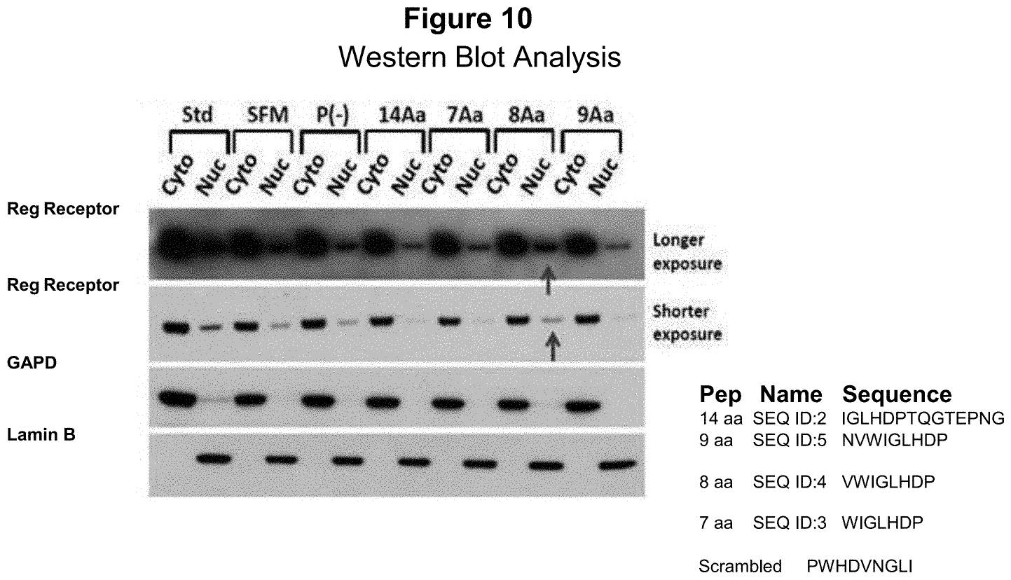

FIG. 10 demonstrates a confirmation study utilizing Western blot analysis to evaluate the translocation of the Reg Receptor from the cytoplasmic membrane of human extra-islet exocrine tissue inclusive of ductal cells containing progenitor. Western blot analysis identified the presence of the Reg receptor on the cytoplasmic membrane and the movement of the Reg Receptor from the cytoplasmic membrane through the cytoplasm to the nucleus in the presence of the Reg peptides (30-minute treatment). Cytoplasmic extracts were obtained in 10 mM HEPES (pH 8.0), 1 mM EDTA, 1.5 mM MgCl2, 10 mM KCl, 0.5 mM DTT, 200 mM sucrose and 0.5% Nonidet P-40. Nuclear extracts were obtained in 20 mM HEPES (pH 7.9), 0.75 mM MgCl2, 210 mM NaCl, 50 mM KCl, 1 mM EDTA, 10% glycerol, and 0.5 mM DTT. Both extraction buffers contained 0.5 mM PMSF, 1 μg/ml leupeptin, 1 μg/ml aprotinin, 2.5 mM Na4P2O7.1 mM 3-glycerophosphate, and 1 mM Na3VO4. Reg Receptor extracts were size fractionated on SDS-polyacrylamide gels and transferred to nitrocellulose. After blocking in 3% milk in Tris-buffered saline (pH 7.4), blots were sequentially incubated with rabbit anti-human Reg Receptor antibody overnight at 4° C. and appropriate horseradish peroxidase-conjugated secondary antibody. Secondary signals were developed with chemiluminescence substrate and analyzed by autoradiography.

Fractions and quality control utilized the fractions with two antibodies, GAPDH as a cytosol molecule and Lamin B is a nuclear molecule. Both GADPH and Lamin B were demonstrated in this invention to serve as excellent controls for the nuclear and cytosolic fractions (FIG. 9). Evaluation was conducted to determine the impact and interaction of the Reg Receptor with the Reg peptides and the human 14-amino acid (SEQ ID NO: 2) peptide. This invention demonstrates in FIG. 10 that the 8-amino acid (SEQ ID NO: 4) Reg resulted in the translocation of the Reg Receptor from the cytoplasm to the nucleus both in the shorter and longer exposure times.

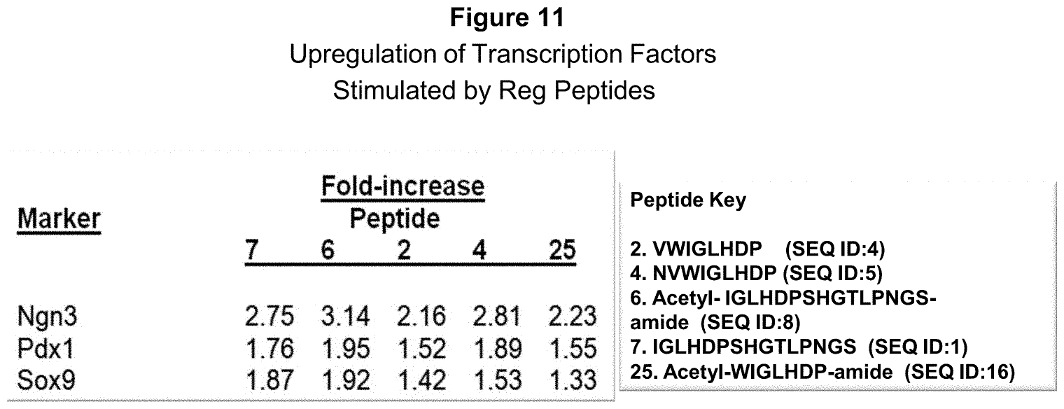

FIG. 11 demonstrates the in vivo efficacy of Reg peptides on pancreatic transcription factors NGN3, Pdx1, and Sox9 compared to uninjected and vehicle alone controls with cohorts of 10 mice (adult, age ˜8 weeks, mixed male and female) will be injected intraperitoneally with selected candidate Reg peptides at 2-3 dosages/animal daily across 2-3 time courses (an average of 60 mice/peptide tested). An additional 20 animals will serve as negative (uninjected and vehicle alone) controls. Pancreata were subjected to rapid dissection and denaturant RNA preparation followed by real-time, quantitative RT-PCR analysis of gene expression markers for beta cell development and differentiation, including Ngn3, Pdx1 and Sox9. All Reg peptides had a significant fold increase in transcription factors ranging from a 1.33 fold rise to a 3.44-fold rise compared to vehicle and control transcription factors, indicating the ability to raise pancreatic transcription factors in mice.

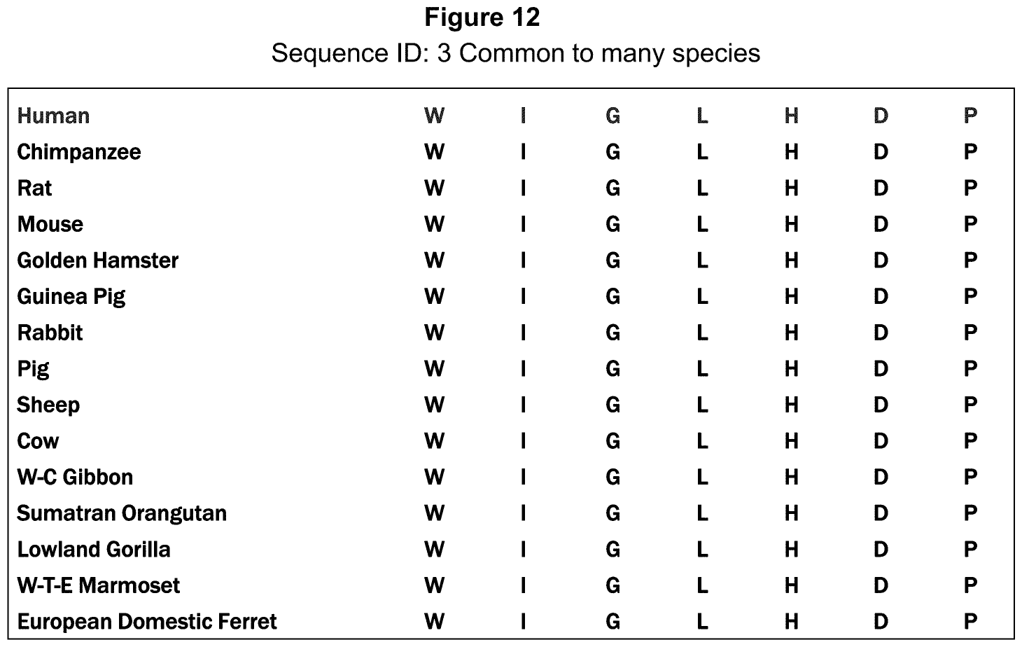

FIG. 12 shows the identification of the Reg Peptide 7-amino acid human Reg sequence (SEQ ID NO: 3) that has 100% homology with sequences found in other mammals including chimpanzee, rat, mouse, golden hamster, guinea pig, rabbit, pig, sheep, cow, white-cheeked gibbon, Sumatran orangutan, Lowland gorilla, white-tufted-eared marmoset, European domestic ferret which has not been described in the prior art as being a peptide that interacts with the Reg receptor that can generate new neurons, cardiac myocytes, nephrons and hepatocytes in times of injury when the Reg receptor is upregulated in such organs. This invention evaluated GenBank, Basic Local Alignment Search Tool (BLAST) algorithm and UniProtKB which produced by the UniProt Consortium which consists of groups from the European Bioinformatics Institute (EBI), the Swiss Institute of Bioinformatics (SIB) and the Protein Information Resource (PIR).

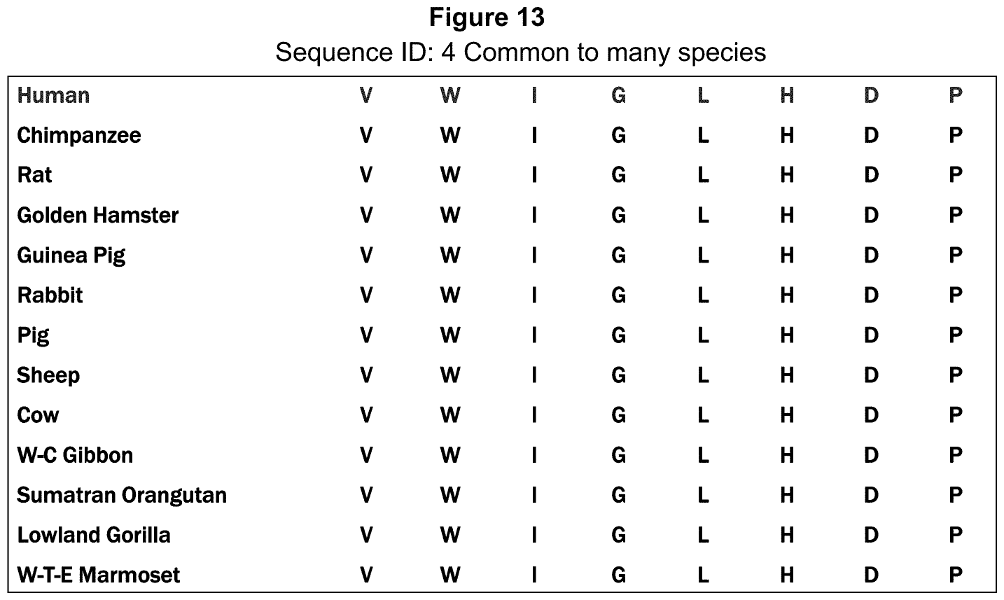

FIG. 13 shows the identification of the Reg 8-amino acid human Reg sequence (SEQ ID NO: 4) that has 100% homology with sequences found in other mammals including chimpanzee, rat, mouse, golden hamster, guinea pig, rabbit, pig, sheep, cow, white-cheeked gibbon, Sumatran orangutan, Lowland gorilla, and white-tufted-eared marmoset which have not been described in the prior art as being a peptide that interacts with the Reg receptor that can generate new neurons, cardiac myocytes, nephrons and hepatocytes. This invention evaluated GenBank, Basic Local Alignment Search Tool (BLAST) algorithm and UniProtKB which produced by the UniProt Consortium which consists of groups from the European Bioinformatics Institute (EBI), the Swiss Institute of Bioinformatics (SIB) and the Protein Information Resource (PIR)

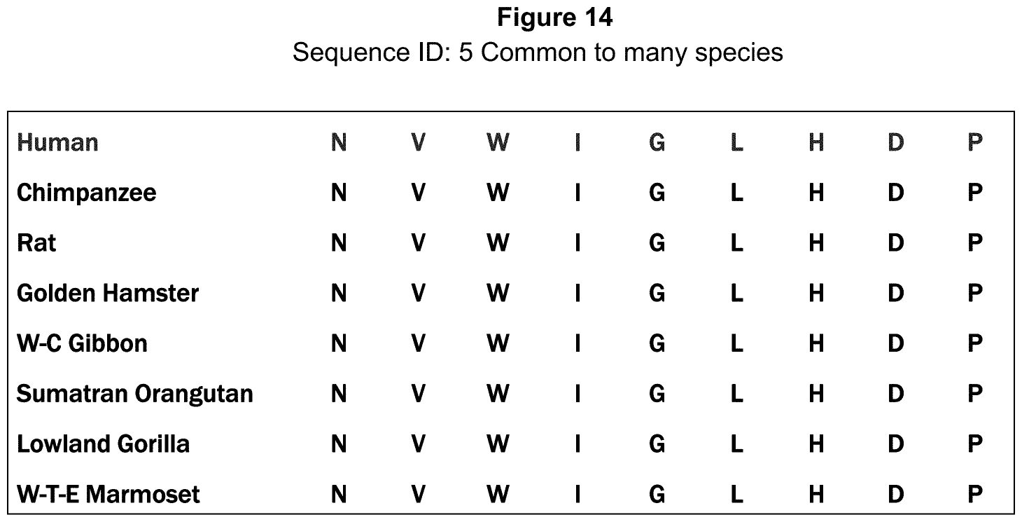

FIG. 14 shows the identification of the Reg 9-amino acid human Reg sequence (SEQ ID NO: 5) that has 100% homology with sequences found in other mammals including chimpanzee, rat, golden hamster, white-cheeked gibbon, Sumatran orangutan, Lowland gorilla, white-tufted-eared marmoset, which have not been described in the prior art as being a peptide that interacts with the Reg receptor that can generate for generation of new neurons, cardiac myocytes, nephrons and hepatocytes due to its ability to bind to This invention evaluated GenBank, Basic Local Alignment Search Tool (BLAST) algorithm and UniProtKB, which produced by the UniProt Consortium which consists of groups from the European Bioinformatics Institute (EBI), the Swiss Institute of Bioinformatics (SIB) and the Protein Information Resource (PIR).

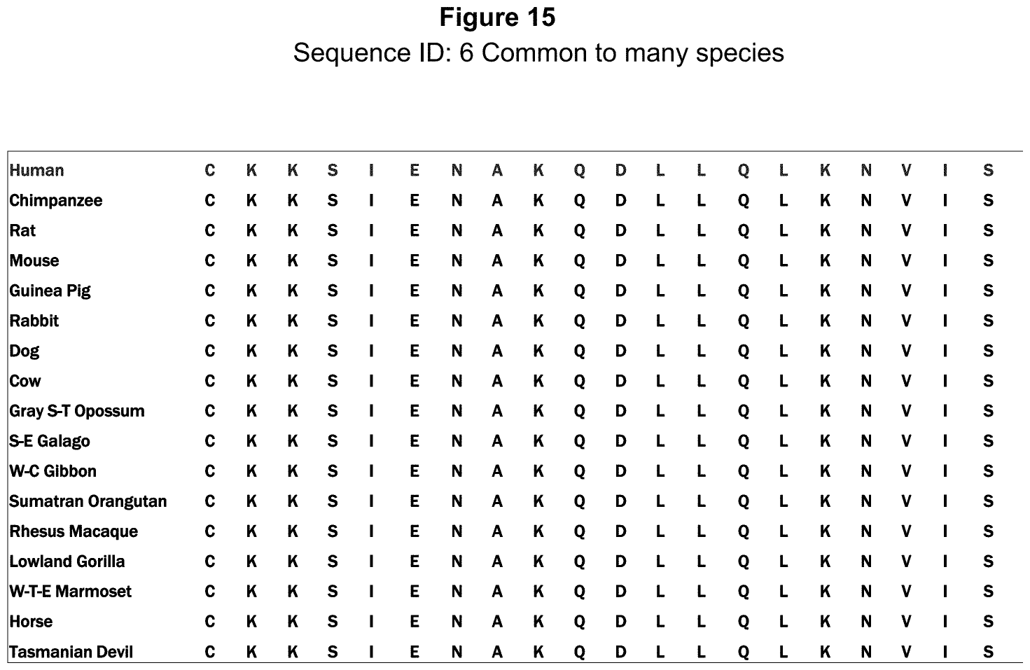

FIG. 15 demonstrates the 20-amino acid human sequence (SEQ ID NO: 6) that has been identified within the human Reg receptor that has 100% homology with Reg receptor sequences found in other mammals including chimp, rat, mouse, guinea pig, rabbit, dog, cow, opossum, galago, white-cheeked-gibbon, Sumatran orangutan, macaque, Lowland gorilla, white-tufted eared marmoset, horse and Tasmanian devil from which stimulatory antibodies and small molecules to this receptor site can be generated to result in new neurons, hepatocytes, myocytes and nephrons. This invention evaluated GenBank, Basic Local Alignment Search Tool (BLAST) algorithm and UniProtKB which produced by the UniProt Consortium which consists of groups from the European Bioinformatics Institute (EBI), the Swiss Institute of Bioinformatics (SIB) and the Protein Information Resource (PIR).

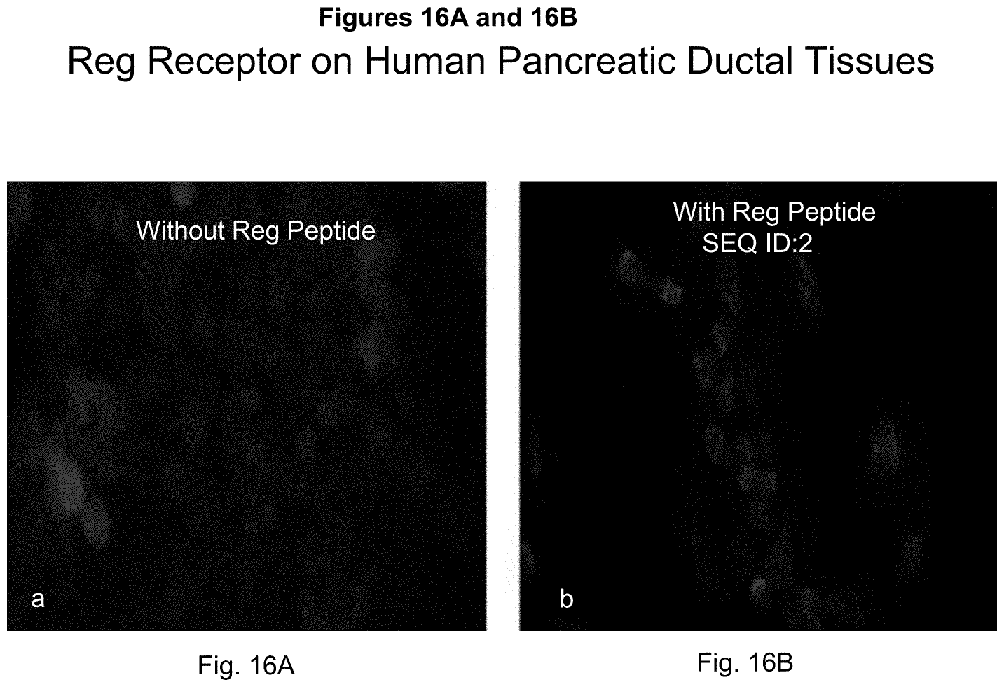

FIGS. 16A and 16B shows immunofluorescent staining of Reg receptor on the cell surface of human pancreatic exocrine ductal cells. In utilizing Cy3 immunofluorescent staining of Reg receptor in human pancreatic ductal cells in standard medium, there is immunofluorescent staining of Reg receptor, which is well-defined at the cell borders indicating surface expression of Reg receptor on the cytoplasmic membrane of cells (FIG. 16A). FIG. 16B demonstrates the difference in Reg receptor staining when cells are exposed to Reg peptide (SEQ ID NO: 2).

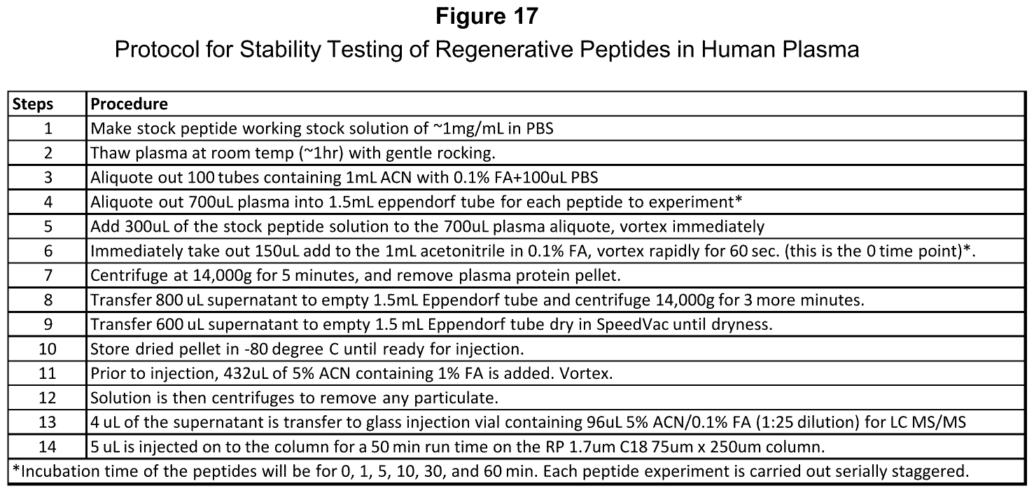

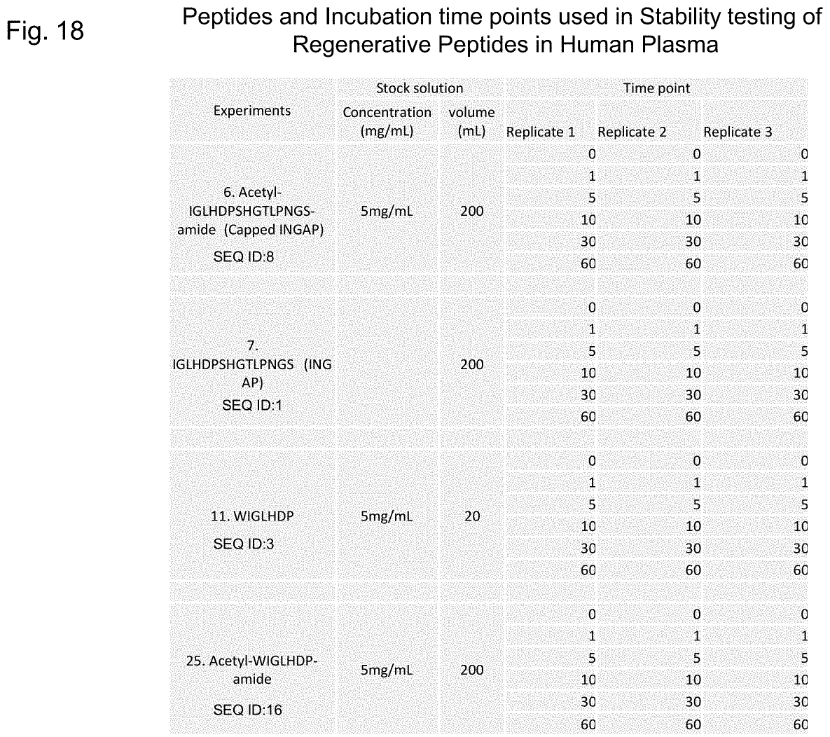

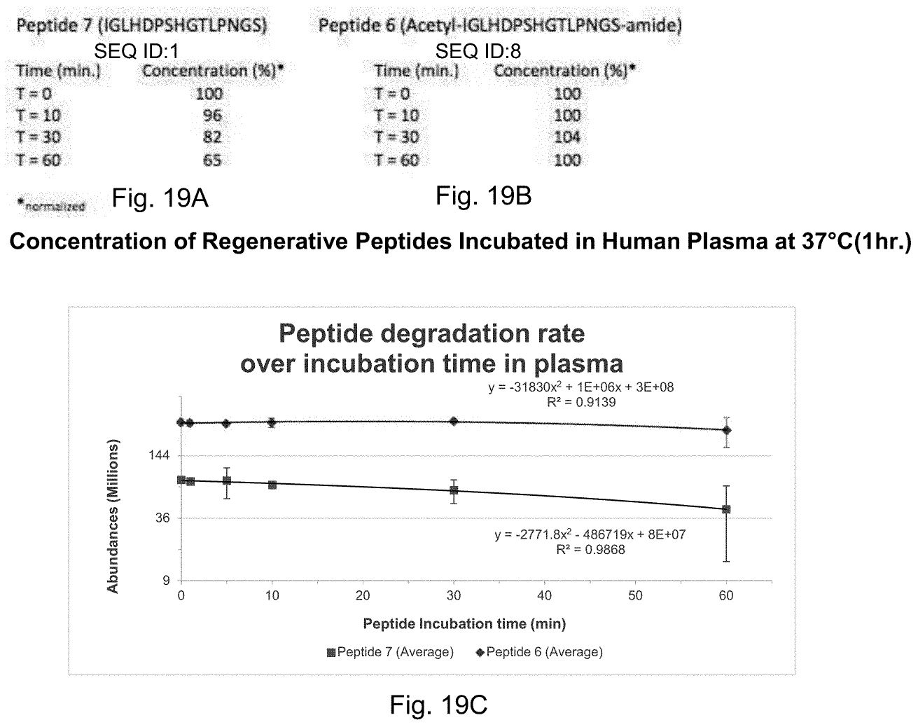

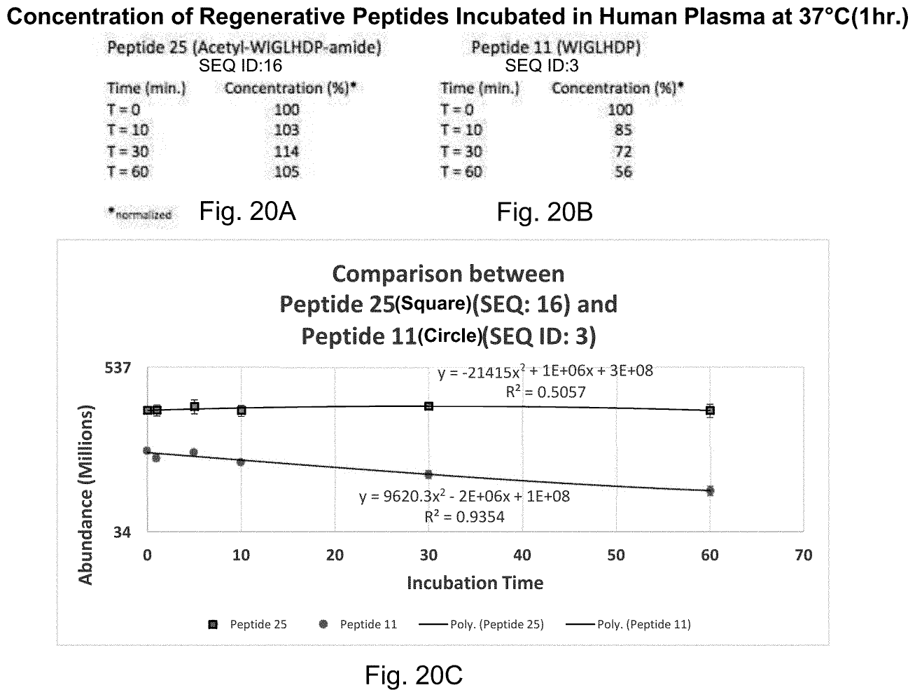

FIGS. 17-20C demonstrate the methods for stability testing of Reg peptides SEQ ID NO: 1, SEQ ID NO: 8, SEQ ID NO: 3 and SEQ ID NO:16 and the incubation time points used in the stability testing in human plasma. FIG. 17 shows the protocol for the stability testing in human plasma for the Regenerative Peptides SEQ ID NO: 1, SEQ ID NO: 8, SEQ ID NO:3 and SEQ ID NO:16. FIG. 17. shows Peptides and Incubation time points used in Stability testing of Regenerative Peptides in Human Plasma. FIGS. 18A-C shows the results of the Regenerative Peptides SEQ ID NO:1 and SEQ ID NO: 8 incubated in human plasma. SEQ ID NO: 8 is significantly more resistant to proteolysis in human plasma compared to SEQ ID NO:1. FIGS. 20A-C show the results of the Regenerative Peptides SEQ ID NO: 16 and SEQ ID NO: 3 incubated in human plasma. SEQ ID NO: 16 is significantly more resistant to proteolysis in human plasma compared to SEQ ID NO: 3.

### BRIEF DESCRIPTION OF THE SEQUENCES

As used herein, “Peg-40KD-maleimide” includes:

### Methoxy-PEG-(CH2)3NHCO(CH2)2-MAL, Mw 40,000

Chemical Name: α-[3-(3-Maleimido-1-oxopropyl)amino]propyl-ω-methoxy, polyoxyethylene CAS #: 883993-35-9

As used herein, 3-Mpa=3-mercaptopropionic acid, CAS #: 107-96-0

### SEQ ID NO: 1

This is the 15-amino acid peptide found within the mammalian Reg protein

SEQ ID NO: 2
Ile-Gly-Leu-His-Asp-Pro-Ser-His-Gly-Thr-Leu-Pro-
Asn-Gly-Ser

This is the 14-amino acid peptide found within the human Reg protein

SEQ ID NO: 3
Ile-Gly-Leu-His-Asp-Pro-Thr-Gln-Gly-Thr-Glu-Pro-
Asn-Gly

This is the 7-amino acid peptide found within the human and mammalian Reg protein

SEQ ID NO: 4
Trp-Ile-Gly-Leu-His-Asp-Pro

This is the 8-amino acid peptide found within the human and mammalian Reg protein

SEQ ID NO: 5
Val-Trp-Ile-Gly-Leu-His-Asp-Pro

This is the 9-amino acid peptide found within the human and mammalian Reg protein

SEQ ID NO: 6
Asn-Val-Trp-Ile-Gly-Leu-His-Asp-Pro

This is a 20-amino acid peptide found within the human Reg receptor also known as EXTL-3

SEQ ID NO: 7
Cys-Lys-Lys-Ser-Ile-Glu-Asn-Ala-Lys-Gln-Asp-Leu-
Leu-Gln-Leu-Lys-Asn-Val-Ile-Ser

Is the 919-amino acid human Reg receptor, also known as EXTL-3, exostosin-like 3 (public accession number NP_001431).

SEQ ID NO: 8
MTGYTMLRNGGAGNGGQTCMLRWSNRIRLTWLSFTLFVILVFFPLIAHY
YLTTLDEADEAGKRIFGPRVGNELCEVKHVLDLCRIRESVSEELLQLEA
KRQELNSEIAKLNLKIEACKKSIENAKQDLLQLKNVISQTEHSYKELMA
QNQPKLSLPMLLPEKDDAGLPPPKATRGCRLHNCFDYSRCPLTSGFPVY
VYDSDQFVFGSYLDPLVKQAFQATARANVYVTENADIACLYVILVGEMQ
EPVVLRPAELEKQLYSLPHWRTDGHNHVIINLSRKSDTQNLLYNVSTGR
AMVAQSTFYTVQYRPGFDLVVSPLVHAMSEPNFMEIPPQVPVKRKYLFT
FQGEKIESLRSSLQEARSFEEEMEGDPPADYDDRIIATLKAVQDSKLDQ
VLVEFTCKNQPKPSLPTEWALCGEREDRLELLKLSTFALIITPGDPRLV
ISSGCATRLFEALEVGAVPVVLGEQVQLPYQDMLQWNEAALVVPKPRVT
EVHFLLRSLSDSDLLAMRRQGRFLWETYFSTADSIFNTVLAMIRTRIQI
PAAPIREEAAAEIPIIRSGKAAGTDPNMADNGDLDLGPVETEPPYASPR
YLRNFTLTVTDFYRSWNCAPGPFHLFPHTPFDPVLPSEAKFLGSGTGFR
PIGGGAGGSGKEFQAALGGNVPREQFTVVMLTYEREEVLMNSLERLNGL
PYLNKVVVVWNSPKLPSEDLLWPDIGVPIMVVRTEKNSLNNRFLPWNEI
ETEAILSIDDDAHLRHDEIMFGFRVWREARDRIVGFPGRYHAWDIPHQS
WLYNSNYSCELSMVLTGAAFFHKYYAYLYSYVMPQAIRDMVDEYINCED
IAMNFLVSHITRKPPIKVTSRWTFRCPGCPQALSHDDSHFRERHKCINF
FVKVYGYMPLLYTQFRVDSVLFKTRLPHDKTKCFKFI

This is the optimization of the mammalian 15-amino acid Reg peptide by blocking with an n-terminal acetyl group and a c-terminal amide group.

SEQ ID NO: 9
Ac--Ile-Gly-Leu-His-Asp-Pro-Ser-His-Gly-Thr-Leu-
Pro-Asn-Gly-Ser-NH2

This is a peglyated version of the mammalian 15-amino acid Reg peptide

SEQ ID NO: 10
Ac--Ile-Gly-Leu-His-Asp-Pro-Ser-His-Gly-Thr-Leu-
Pro-Asn-Gly-Ser (Peg-40KD-maleimide)-NH2

This is a peglyated version of the mammalian 15-amino acid Reg peptide

SEQ ID NO: 16
Peg-40KD-maleimide-3-Mpa-Ile-Gly-Leu-His-Asp-Pro-
Ser-His-Gly-Thr-Leu-Pro-Asn-Gly-Ser-NH2
(3-Mpa = 3-mercaptopropionic acid)

This is a cyclized versions of the mammalian 15-amino acid Reg peptide to include but are not limited to:

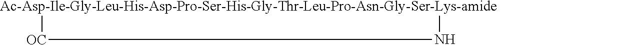

Cyclic amide bond between side chain of Asp on position 1 and Lys on position 17

### SEQ ID NO: 12

This is the optimization of the 14-amino acid human Reg peptide by blocking with an n-terminal acetyl group and a c-terminal amide group.

SEQ ID NO: 13
Ac--Ile-Gly-Leu-His-Asp-Pro-Thr-Gln-Gly-Thr-Glu-
Pro-Asn-Gly--NH2

This is a peglyated version of the human 14-amino acid Reg peptide

SEQ ID NO: 14
Ac--Ile-Gly-Leu-His-Asp-Pro-Thr-Gln-Gly-Thr-G1u-
Pro-Asn-Gly (Peg-40KD-maleimide)-NH2

This is a peglyated version of the human 14-amino acid Reg peptide

SEQ ID NO: 15
Peg-40KD-maleimide-3-Mpa-Ile-Gly-Leu-His-Asp-Pro-
Thr-Gln-Gly-Thr-G1u-Pro-Asn-Gly-NH2
(3-Mpa = 3-mercaptopropionic acid)

Cyclized versions of the human 14-amino acid Reg peptide to include but are not limited to:

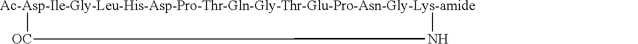

Cyclic amide bond between side chain of Asp on position 1 and Lys on position 16

### SEQ ID NO: 16

This is the optimization of the 7-amino acid human and mammalian Reg peptide by blocking with an n-terminal acetyl group and a c-terminal amide group.

SEQ ID NO: 17
Ac--Trp-Ile-Gly-Leu-His-Asp-Pro--NH2

This is a peglyated version of the human and mammalian 7-amino acid Reg peptide

SEQ ID NO: 18
Ac-Trp-Ile-Gly-Leu-His-Asp-Pro (Peg-40KD-
maleimide)-NH2

This is a peglyated version of the human and mammalian 7-amino acid Reg peptide

SEQ ID NO: 19
Peg-40KD-maleimide-3-Mpa-Trp-Ile-Gly-Leu-His-Asp-
Pro-NH2
(3-Mpa = 3-mercaptopropionic acid)

Cyclized versions of the human and mammalian 7-amino acid Reg 3 peptide to include but are not limited to:

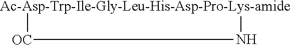

Cyclic amide bond between side chain of Asp on position 1 and Lys on position 9

### SEQ ID NO: 20

This is the optimization of the 8-amino acid human and mammalian Reg peptide by blocking with an n-terminal acetyl group and a c-terminal amide group.

SEQ ID NO: 21
Ac-Val-Trp-Ile-Gly-Leu-His-Asp-Pro--NH2

This is a peglyated version of the human and mammalian 8-amino acid Reg protein

SEQ ID NO: 22
Ac-Val-Trp-Ile-Gly-Leu-His-Asp-Pro (Peg-40KD-
maleimide)-NH2

This is a peglyated version of the human and mammalian 8-amino acid Reg peptide

SEQ ID NO: 23
Peg-40KD-maleimide-3-Mpa-Val-Trp-Ile-Gly-Leu-His-
Asp-Pro-NH2
(3-Mpa = 3-mercaptopropionic acid)

Cyclized versions of the human and mammalian 8-amino acid Reg peptide to include but are not limited to:

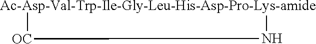

Cyclic amide bond between side chain of Asp on position 1 and Lys on position 10

### SEQ ID NO: 24

This is the optimization of the human and mammalian 9-amino acid Reg peptide by blocking with an n-terminal acetyl group and a c-terminal amide group.

SEQ ID NO: 25
Ac-Asn-Val-Trp-Ile-Gly-Leu-His-Asp-Pro--NH2

This is a peglyated version of the human and mammalian 9-amino acid Reg peptide

SEQ ID NO: 26
Ac-Asn-Val-Trp-Ile-Gly-Leu-His-Asp-Pro (Peg-40KD-
maleimide)-NH2

This is a peglyated version of the human and mammalian 9-amino acid Reg peptide

SEQ ID NO: 27
Peg-40KD-maleimide-3-Mpa-Asn-Val-Trp-Ile-Gly-Leu-
His-Asp-Pro-NH2
(3-Mpa = 3-mercaptopropionic acid)

Cyclized versions of the human and mammalian 9-amino acid Reg peptide to include but are not limited to:

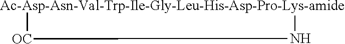

Cyclic amide bond between side chain of Asp on position 1 and Lys on position 11

The Reg peptides may be produced through recombinant molecular biology techniques or solid phase synthesis techniques. Recombinant molecular biology techniques include those described in Molecular Cloning: A Laboratory Manual, Green and Sanbrook, 2012. Solid-phase synthesis techniques are described in Merrifield, in J. Am. Chem. Soc., 15:2149-2154 (1963), M. Bodanszky et al., (1976) Peptide Synthesis, John Wiley & Sons, 2d Ed.; Kent and Clark-Lewis in Synthetic Peptides in Biology and Medicine, p. 295-358, eds. Alitalo, K., et al. Science Publishers, (Amsterdam, 1985); as well as other reference works known to those skilled in the art such. A summary of peptide synthesis techniques may be found in J. Stuart and J. D. Young, Solid Phase Peptide Synthelia, Pierce Chemical Company, Rockford, Ill. (1984), which is incorporated herein by reference. The synthesis of peptides by solution methods may also be used, as described in The Proteins, Vol. II, 3d Ed., p. 105-237, Neurath, H. et al., Eds., Academic Press, New York, N.Y. (1976). Appropriate protective groups for use in such syntheses will be found in the above texts, as well as in J. F. W. McOmie, Protective Groups in Organic Chemistry, Plenum Press, New York, N.Y. (1973), which is incorporated herein by reference. In general, these synthetic methods involve the sequential addition of one or more amino acid residues or protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine. Block synthesis techniques may also be applied to both the solid phase and solution methods of peptide synthesis. Rather than sequential addition of single amino acid residues, preformed blocks comprising two or more amino acid residues in sequence are used as either starting subunits or subsequently added units rather than single amino acid residues. Alternative or additional peptide synthesis methods and techniques can be found in Peptide Chemistry: A Practical Textbook: 2nd Edition, Miklos Bodanszky, 1993.

Reg peptides of the invention may also be synthesized by solid-phase peptide synthesis using procedures similar to those described by Merrifield, 1963, J. Am. Chem. Soc., 85:2149. During synthesis, N-a-protected amino acids having protected side chains are added stepwise to a growing polypeptide chain linked by its C-terminal and to an insoluble polymeric support, i.e., polystyrene beads. The proteins are synthesized by linking an amino group of an N-a-deprotected amino acid to an a-carboxyl group of an N-a-protected amino acid that has been activated by reacting it with a reagent such as dicyclohexylcarbodiimide. The attachment of a free amino group to the activated carboxyl leads to peptide bond formation. The most commonly used N-a-protecting groups include Boc, which is acid labile, and Fmoc, which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting peptides is accomplished using conventional procedures, such as preparative HPLC using gel permeation, partition and/or ion exchange chromatography. The choice of appropriate matrices and buffers are well known in the art and so are not described in detail herein.

Inert polymer molecules such as high molecular weight polyethyleneglycol (PEG) can be attached to a peptide of this disclosure or an analog or derivative thereof with or without a multifunctional linker either through site-specific conjugation of the PEG to the N- or C-terminus of the protein or via epsilon-amino groups present on lysine residues. Linear or branched polymer derivatization that results in minimal loss of biological activity can be used. The degree of conjugation can be closely monitored by SDS-PAGE and mass spectrometry to ensure proper conjugation of PEG molecules. Unreacted PEG can be separated from peptide-PEG conjugates by size-exclusion or by ion-exchange chromatography.

Protocols for blocking peptides with acetyl and amide groups are known in the art and can be found in a number of protein protocol textbooks known in the art. Specific examples include those published in Methods in Molecular Biology, Vol. 35: Peptide Synthesis Protocols, Chapter 8: Site-Specific Chemical Modification Procedures, Edited by M W Pennington and B M Dunn, 1994, as well as U.S. Pat. Nos. 4,708,934, 5,503,989, U.S. Patent Application Publication No. US 20060127995. Alternative or additional protein modification procedures can be found in Peptide Chemistry: A Practical Textbook: 2nd Edition, Miklos Bodanszky, 1993. Further, peptide cyclization (Davies, J. Peptide Sci. 9: 471-501 (2003)) and pegylation (Roberts, Advanced Drug Delivery Reviews (2002) 54:459-476 and Veronese, Biomaterials (2001) 22(5):405-17) methods have been reviewed. Protocols for creating maleimide-activated PEG constructs may be found in Schumacher et al., In Situ Maleimide Bridging of Disulfides and a New Approach to Protein PEGylation, Bioconjugate Chem., 2011, 22 (2), pp 132-136, Doherty et al., Site-Specific PEGylation of Engineered Cysteine Analogs of Recombinant Human Granulocyte-Macrophage Colony-Stimulating Factor, Bioconjug Chem. 2005; 16(5): 1291-1298, US Patent Application Publication No. 20090298746 A1, European Patent No. EP 1881850 B1, European Patent No. EP 2178900 B1.

### EMBODIMENTS

Embodiments of the invention include but are not limited to the following:

### Embodiment 1A

Regenerative peptides which comprise the following 7-15-amino acid Reg sequences and 7-15-amino acid optimized Reg Peptides sequences from the human and mammalian Reg peptides that are utilized for both direct production of nerve cells, cardiac myocytes, liver cells via in vivo formation by the usage of the 7-15 optimized and native peptides in times of acute loss of cells from the brain, spinal cord, heat and liver with such peptides used for the formation of new neurons, including motor neurons, cardiac myocytes, liver hepatocytes and include: SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27 and may be delivered to a patient in need of new neurons, myocytes, hepatocytes by routes of delivery of therapies described include, but are not limited to oral, intravenous, intra-arterial, subcutaneous delivery and intrathecal delivery and through the spinal canal for generation central neurons and for spinal cord injuries. Also, therapy may be given by organ specific targeting and may include direct administration liver via the umbilical and hepatic artery, femoral or radial arterial delivery to the heart or directly to the arteries supplying the heart, as is done in cardiac catheterizations, and intrathecal delivery and through the spinal canal for central nervous system as is done for antibiotic delivery for central nervous system infections and for chemotherapy delivery for central lesions

### Embodiment 2A

Reg peptides which comprise the following 7-15-amino acid Reg sequences and 7-15-amino acid optimized Reg Peptides sequences from the human and mammalian Reg peptides that are utilized for production of nerve cells, cardiac myocytes, liver cells and cells via ex vivo formation by the usage of the 7-15 optimized and native peptides generated outside of the body using progenitor cells or tissue from organs expressing Reg receptors and can be transformed by human and mammalian 7-15-amino acid Reg peptide sequences into functional neurons, myocytes, hepatocytes for usage in times of acute loss of cells from the brain, spinal cord, heart, and liver with such peptides used for the formation of new neurons, including motor neurons, cardiac myocytes, liver hepatocytes or tissue from organs expressing the Reg receptor and include: SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27 and then neurons, myocytes, nephrons, hepatocytes may be delivered to a patient in need of new neurons, myocytes, hepatocytes, and nephrons by routes of delivery of therapies described include, but are not limited to oral, intravenous, intra-arterial, subcutaneous delivery and intrathecal delivery and through the spinal canal for generation central neurons and for spinal cord injuries.

Also, therapy may be given by organ specific targeting and may include direct administration liver via the umbilical and hepatic artery, femoral or radial arterial delivery to the heart or directly to the arteries supplying the heart, as is done in cardiac catheterizations, and intrathecal delivery and through the spinal canal for central nervous system as is done for antibiotic delivery for central nervous system infections and for chemotherapy delivery for central lesions and delivered to the renal artery via the femoral artery with a small puncture in the groin. Once the catheter is positioned in the artery or vein supplying blood to the heart, liver or targeted to brain, above sequences can be targeted to the organ of injury expressing the Reg receptor.

This modality of delivery includes, but is not limited to oral, intravenous, subcutaneously, intra-arterial, intrathecal and into the spinal column and targeted delivery to the brain, spinal column, liver or delivered directly or indirectly to the brain, spinal column, heart and liver and may include targeted therapy to the given organ, which may be delivered orally, intravenously, intra-arterially, intrathecally or directly into the spinal column at the site of injury.

### Embodiment 3A

Peptidomimetics, small molecules and stimulatory antibodies used in vivo to stimulate the 20-amino acid region on the 919-amino acid Reg receptor/EXTL-3 (SEQ: ID 6) to generate of new neurons, cardiac myocytes, and hepatocytes. Administration of peptidomimetics includes formulations that stimulate the Reg receptor resulting in formation of new cardiac myocytes, hepatocytes and neurons. This modality of delivery includes, but is not limited to: oral, intravenous, subcutaneously, intraarterial, intrathecal and into the spinal column and targeted delivery to the brain, spinal column, liver or delivered directly or indirectly to the brain, spinal column, heart and liver and may include targeted therapy to the given organ, which may be delivered orally, intravenously, intra-arterially, intrathecally or directly into the spinal column at the site of injury.

### Embodiment 4A

Peptidomimetics, small molecules and stimulatory antibodies used ex vivo to stimulate the 20-amino acid region on the 919-amino acid Reg receptor (SEQ: ID 6) to generate of new neurons, cardiac myocytes, nephrons and hepatocytes to transform progenitor cells from organs that express Reg receptors. The modality of delivery of peptidomimetics, small molecules and antibodies to stimulate (SEQ: ID 6) includes, but is not limited to: oral, intravenous, subcutaneously, intra-arterial, intrathecal and into the spinal column and targeted delivery to the brain, spinal column, liver or delivered directly or indirectly to the brain, spinal column, heart and liver and may include targeted therapy to the given organ, which may be delivered orally, intravenously, intra-arterially, intrathecally or directly into the spinal column at the site of injury.

### Embodiment 5A

SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27 are used to generate neurons, hepatocytes or myocytes in vivo and ex vivo via generation of tissue from progenitor cells or tissue from organs expressing Reg receptors and may be used in conjunction with other medications used to treat heart disease, liver disease, neurological diseases and or diseases of organs expressing the Reg receptor.

### Embodiment 6A

Peptidomimetics, small molecules and stimulatory antibodies used ex vivo to stimulate the 20-amino acid region on the 919-amino acid Reg Receptor/EXTL-3 (SEQ: ID 6) to generate of new neurons, cardiac myocytes, and hepatocytes either in vivo or ex-vivo and may be used in conjunction with other medications used to treat heart disease, liver disease, neurological diseases and renal diseases.

### Embodiment 1B

An isolated or modified peptide having an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

### Embodiment 2B

A pharmaceutical formulation comprising the peptide of embodiment 1B.

### Embodiment 3B

The pharmaceutical formulation of embodiment 2B, wherein the formulation is a soluble liposome or nanoparticle preparation.

### Embodiment 4B

The pharmaceutical formulation of embodiment 2B, wherein the formulation comprises a targeting agent for targeted administration to heart, brain, spinal column, liver.

### Embodiment 5B

A method of treating a subject in need of one more differentiated cells or tissue types, comprising administering to the subject a peptide having Reg receptor binding activity, wherein the amount of peptide is effective for forming differentiated cells or tissues in the subject in vivo.

### Embodiment 6B

The method of embodiment 5B, wherein the peptide having Reg Receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

### Embodiment 7B

The method of embodiment 5B, wherein the one or more cell or tissue types are heart, liver, brain, spinal column, or cells expressing the Reg receptor

### Embodiment 8B

The method of embodiment 5B, wherein the peptide is administered directly to the heart, liver, brain or spinal cord of a subject.

### Embodiment 9B

The method of embodiment 5B, wherein the peptide is administered by way of intravenous, subcutaneous, intra-arterial, or intrathecal delivery.

### Embodiment 10B

The method of embodiment 5B, wherein the subject has a condition selected from the group consisting of heart disease, myocardial infarction, stroke, acute brain injury, neurodegenerative disease, spinal cord injury, peripheral neuropathy, liver disease or injured organs expressing the Reg receptor.

### Embodiment 11B

The method of transforming progenitor cells to differentiated tissue cells, comprising:

culturing progenitor cells ex vivo; or tissue of organs expressing the Reg receptor

contacting progenitor cells or tissue expressing the Reg receptor with a peptide having Reg receptor binding activity,

wherein the amount of peptide is effective for transforming progenitor cells or tissue from organs expressing the Reg receptor into differentiated cells.

### Embodiment 12B

The method of embodiment 11B, wherein the peptide having Reg Receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

### Embodiment 13B

The method of embodiment 11B, wherein progenitor cells are selected from the group consisting of neural stems cells, tissue stem cells, progenitor cells or tissue from organs expressing Reg receptors.

### Embodiment 14B

The method of embodiment 11B, wherein the differentiated tissue cells are selected from the group consisting of brain, spinal cord, heart, and liver tissue cells.

### Embodiment 15B

A method of treating a subject in need of one more differentiated cells or tissues, the method comprising:
progenitor cells or tissue from organs expressing Reg receptors ex vivo;
contacting progenitor cells or tissue expressing the Reg receptor with a peptide having Reg Receptor binding activity, wherein the amount of peptide is effective for transforming progenitor cells to differentiated cells or tissues in culture; and administering the one or more differentiated cells or tissues to the subject.

### Embodiment 16B

The method of embodiment 15B, wherein the peptide having Reg Receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

### Embodiment 17B

The method of embodiment 15B, wherein progenitor cells or tissue from organs expressing Reg receptors

### Embodiment 18B

The method of embodiment 15B, wherein the differentiated cells or tissues are selected from the group consisting of brain, spinal cord, heart, liver cells or organs expressing the Reg receptor.

### Embodiment 19B

The method of embodiment 15B, wherein the differentiated cells are administered directly to the heart, liver, brain, spinal cord of a subject.

### Embodiment 20B

The method of embodiment 15B, wherein the subject has a condition selected from the group consisting of heart disease, myocardial infarction, stroke, acute brain injury, neurodegenerative disease, spinal cord injury, peripheral neuropathy, acute and liver disease or conditions affecting organs that express the Reg receptor.

### EXAMPLES

The following examples serve to further illustrate the invention and should not be used to limit the invention.

### Example 1

A patient presents with an acute myocardial infarction as evidenced by EKG and cardiac isoenzymes. The patient undergoes cardiac catheterization and prior to injection of contrast is given 60 mg of SEQ ID NO: 8, which will be delivered into the myocardium and bind to the upregulated Reg receptor in the location of the acute injury.

### Example 2

A patient suffers a neck injury from being thrown from a horse with the spinal cord severed at cervical disc 7 and has immediate paralysis. Sixty mg of SEQ ID NO: 8, is delivered directly via injection to the severed cord under fluoroscopy and delivered every day for 14 days.

### Example 3

A patient undergoes a large resection of the liver for removal of a large metastatic lesion from colon cancer. An oral hepatic targeted nanoparticle encapsulation of SEQ ID NO: 8 in a dosage of 60 mg and is given to the patient twice daily for generation of new hepatic cells.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure including published patents, published patent applications, books, and journal articles are each individually incorporated by reference herein in their entireties and as such are intended to provide an efficient way of supplementing the enabling disclosure of this invention as well as provide background detailing the level of ordinary skill in the art.

1. An isolated or modified peptide having an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

2. A pharmaceutical formulation comprising the peptide of claim 1.

3. The pharmaceutical formulation of claim 2, wherein the formulation is a soluble liposome or nanoparticle preparation.

4. The pharmaceutical formulation of claim 2, wherein the formulation comprises a targeting agent for targeted administration to heart, brain, spinal column, liver or organ that is injured and expresses the Reg receptor.

5. A method of treating a subject in need of one or more differentiated cells or tissue types, comprising administering to the subject a Reg peptide or optimized Reg peptide with binding activity to the Reg receptor, wherein the amount of peptide is effective for forming differentiated cells or tissues from progenitor cells in the subject in vivo.

6. The method of claim 5, wherein the peptide having Reg receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

7. The method of claim 5, wherein the tissue types are heart, liver, brain, spinal cord neuron, sensory or motor neuron, peripheral neuron, liver or tissues from organs expressing the Reg receptor.

8. The method of claim 5, wherein the peptide is administered directly for usage in an acute organ injury of the heart, liver, brain, spinal cord, or organ which expresses the Reg receptor.

9. The method of claim 5, wherein the peptide is administered by way of intravenous, subcutaneous, intra-arterial, or intrathecal delivery.

10. The method of claim 5, wherein the subject has a condition selected from the group consisting of heart disease, myocardial infarction, stroke, acute brain injury, neurodegenerative disease, spinal cord injury, peripheral neuropathy, liver disease or liver failure, acute and chronic organ disease from an organ expressing the Reg receptor.

11. The method of transforming progenitor cells or from tissues from organs expressing Reg receptors, comprising:
culturing progenitor cells or cells from an organ expressing the Reg receptor ex vivo; and
contacting progenitor cells or tissue from an organ expressing Reg receptor with a peptide having Reg Receptor binding activity,
wherein the amount of peptide is effective for transforming progenitor cells to differentiated cells or cells or tissues from an organ expressing Reg receptors.

12. The method of claim 11, wherein the peptide having Reg receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

13. The method of claim 11, wherein progenitor cells or cells or tissue from organs expressing Reg receptors are selected.

14. The method of claim 11, wherein the differentiated cells or tissues are selected from the group consisting of brain, spinal cord, heart, and liver cells or organs which express the Reg receptor.

15. A method of treating a subject in need of one more differentiated cells or tissues, the method comprising:
culturing progenitor cells or cells or tissues from an organ or organs expressing the Reg receptor ex vivo;
contacting progenitor cells or cells or tissue from an organ expressing the Reg receptor with a peptide having Reg receptor binding activity, wherein the amount of peptide is effective for transforming progenitor cells or cells or tissues from organs expressing the Reg receptor into new cells and administering the one or more differentiated cells or tissues to the subject.

16. The method of claim 15, wherein the peptide having Reg receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

17. The method of claim 15, wherein the cells are progenitor cells or cells or tissue expressing the Reg receptor.

18. The method of claim 15, wherein the cells or tissues are selected from the group consisting of brain, spinal cord, heart and liver cells or tissues or cells from organs expressing the Reg receptor.

19. The method of claim 15, wherein the differentiated cells are administered directly to the heart, liver, brain, spinal cord or injured organ that expresses the Reg receptor in a subject.

20. The method of claim 15, wherein the subject has a condition selected from the group consisting of heart disease, myocardial infarction, stroke, acute brain injury, neurodegenerative disease, spinal cord injury, peripheral neuropathy, liver disease or diseases of organs, which express the Reg receptor.
